(19) United States
(12) Patent Application Publication
(10) Pub. No.: US 2012/0046246 A1
(43) Pub. Date: Feb. 23, 2012

(54) METHODS FOR TREATING OSTEOCLAST-RELATED DISEASE, COMPOUNDS AND COMPOSITIONS THEREOF

(75) Inventors: Lexie Shannon Holliday, Gainesville, FL (US); David A. Ostrov, Gainesville, FL (US)

(73) Assignee: University of Florida Research Foundation, Gainesville, FL (US)

(21) Appl. No.: 13/140,584

(22) PCT Filed: Dec. 18, 2009
(86) PCT No.: PCT/US09/68793

§ 371 Date: Oct. 28, 2011

Related U.S. Application Data
(60) Provisional application No. 61/203,267, filed on Dec. 19, 2008.

(51) Int. Cl. A61K 31/675; A61P 19/08; A61P 19/10; A61P 29/00; A61P 19/02; A61K 31/53; A61P 5/30; A61P 31/04; C07D 471/04; C07F 9/6561; C07D 401/10; C07D 487/18; A61K 31/496; A61P 35/00

(52) U.S. Cl. 514/81; 514/253.04; 544/362; 544/337; 544/363; 514/253.08; 544/185; 514/244

(57) ABSTRACT

This invention relates to therapeutic methods for treating or preventing an osteoclast-related disease or disorder in a subject identified as in need of a treatment of bone diseases, compounds for such uses, and compositions thereof.

### BACKGROUND OF THE INVENTION

Osteoporosis is a major health problem that is causing ever more morbidity and mortality in the aging population (Raisz, L. G., Journal of Clinical Investigation, 2005, 115:3318-3325). It involves a gradual reduction of bone density and strength due to excess bone resorption by osteoclasts compared with the amount of bone formation by osteoblasts (Poole, K. E. et al. Osteoporosis and its management. 2006, BM/333:125 1-1256). This ultimately leads to fractures resulting from minimal insults. Currently it is estimated that $18 billion per year is spent for treatment associated with osteoporotic fractures. Unless better ways to treat osteoporosis emerge, as the “baby boomer” generation reaches their retirement years, osteoporotic fractures are a cause of great suffering and will increasingly burden on our health care system.

Common existing drugs used in treatments for osteoporosis, for example, bisphosphonates, calcitonin, and estrogen-replacement, act to block osteoclast formation and induce osteoclasts to undergo programmed cell death (Reszka, A. A. et al. Mini. Rev. Med. Chem. 2004, 4:711-719; Tanaka, S., T. et al. Ann. N.Y. Acad. Sci. 2006, 1068:180-186). Most of these drugs prevent bone loss, but do not restore or repair bone that is already compromised, and therefore they are not anabolic.

During the past two decades, progress has been made in characterizing the molecular underpinnings of osteoclast function (Teitelbaum, S. L. Am. J. Pathol. 2007, 170:427-435). Nevertheless, to date relatively few new therapeutic tools have emerged based on rational strategies making use of the new knowledge. As such, there is an urgent need for improved therapeutic agents that have the capacity to both stop excess bone resorption and trigger new bone formation to repair bone weakened by the disease state.

### SUMMARY OF THE INVENTION

In one aspect, the invention provides a method for treating or preventing an osteoclast-related disease or disorder in a subject identified as in need of a treatment of bone diseases. The method comprises administering to the subject an effective amount of a compound identified as capable of reducing bone resorption and stimulating new bone formation, such that the osteoclast-related disease or disorder is treated or prevented.

In one embodiment, the compound administered to the subject is capable of reducing bone resorption and stimulating new bone formation through inhibiting (or interfering with) interaction between vacuolar H⁺ ATPase (V-ATPase) and F-actin in the membranes of osteoclasts. In another embodiment, the compound is also capable of stimulating microRNA activity. Still in another embodiment, the method includes administering to the subject an effective amount of an additional therapeutic agent selected from the group of bisphosphonates, calcitonin, intermittent PTH, Denosumib and mixtures thereof.

Another aspect of the invention provides a method for identifying a compound for treating or preventing an osteoclast-related disease or disorder in a subject identified as in need of the treatment of bone diseases, the method comprising a) designing computer-based screen to identify a compound that is membrane-permeable, wherein the compound is also capable of binding the actin binding site of V-ATPase; and b) testing the compound for its ability to inhibit (or interfere with) interaction between recombinant V-ATPase and F-actin in vitro or in vivo. In one embodiment, the method further comprises assessing the compound for its ability to disrupt osteoclast bone resorption in vitro cell culture and/or testing the compound for effects on bone loss in vivo. Compounds identified through the afore-described methods are capable of reducing bone resorption and stimulating new bone formation. In one embodiment, the compound being identified is capable of reducing bone resorption and stimulating new bone formation through inhibiting (or interfering with) interaction between V-ATPase and F-actin in the membranes of osteoclasts.

In another aspect, the invention provides a kit for treating or preventing an osteoclast-related disease in a subject identified as in need of the treatment of bone diseases. The kit includes a compound herein, pharmaceutically acceptable esters, salts, and prodrugs thereof, and instructions for use. In further aspects, the invention provides kits for treating or preventing an osteoclast-related disease or disorder in a subject identified as in need of the treatment of bone diseases, assessing the efficacy of the treatment in a subject, monitoring the progress of the subject being treated with a compound herein, identifying or selecting a subject with an osteoclast-related bone disease or disorder for the treatment with a compound herein, and/or treating a subject suffering from or susceptible to an osteoclast-related bone disease or disorder. In one embodiment, the invention provides: a kit comprises a compound capable of reducing bone resorption and stimulating new bone formation through inhibiting (or interfering with) interaction between V-ATPase and F-actin in the membranes of osteoclasts.

In still another aspect, the invention provides methods for treating or preventing an osteoclast-related disease or disorder in a subject identified as in need of a treatment of bone diseases, wherein the methods include identifying or selecting such a subject suffering from or susceptible to an osteoclast-related bone disease or disorder for the treatment with a compound of the invention, (optionally) monitoring the progress of the subject being treated, and (optionally) assessing the efficacy of the treatment in the subject.

In another aspect, the invention provides a packaged composition including an effective amount of a compound identified as capable of reducing bone resorption and stimulating new bone formation, and a pharmaceutically acceptable carrier or diluent. In one embodiment, the compound acts through inhibiting (or interfering with) interaction between V-ATPase and F-actin in the membranes of osteoclasts. The composition may be formulated for treating a subject suffered from or susceptible to an osteoclast-related bone disease or disorder, and packaged with instructions to treat a subject with such a need.

Another aspect of the invention provides a pharmaceutical composition for treating or preventing an osteoclast-related disease or disorder in a subject identified as in need of the treatment of bone diseases. The composition includes an effective amount of a compound identified as capable of reducing bone resorption and stimulating new bone formation, together with a pharmaceutically acceptable carrier. One embodiment provides that the compound is capable of reducing bone resorption and stimulating new bone formation through inhibiting (or interfering with) interaction between V-ATPase and F-actin in the membranes of osteoclasts.

The invention also provides methods for designing, evaluating and identifying a compound that is capable of reducing bone resorption and stimulating new bone formation. Other embodiments of the invention are disclosed infra.

### BRIEF DESCRIPTION OF THE DRAWINGS

The invention is further described below with reference to the following non-limiting examples and with reference to the following figures, in which:

FIG. 1 depicts V-ATPase packed ruffled membrane. In an unactivated osteoclast on a coverslip (left panel), V-ATPases are found in discrete vesicles that are diffusely distributed throughout the cytosol, whereas they are somewhat concentrated around nuclei. In resorbing osteoclasts on a bone slice (right panel), V-ATPases are transported to ruffled membranes, discrete domains of the plasma membrane that appose the bone. Osteoclasts were stained with an anti-E-subunit polyclonal antibody in both panels and were photographed at same magnification in both panels;

FIG. 2 depicts subunits of V-ATPase (right panel) and of F-ATPase (left panel). V-ATPase and of F-ATPase are related enzymes. V-ATPase is composed of numerous subunits, some which have cell type specific isoforms;

FIG. 3 shows that small molecules (as shown, Binhib 2) docked in most favorable location into the actin binding site of subunit B2. The structure of the actin binding site was predicted from the crystal structure of the alpha subunit of F-ATPase;

FIG. 4 depicts a small molecule (depicted as a star) that binds the actin binding site on subunit B of the V-ATPase;

FIG. 5 is a graph showing that enoxacin inhibits osteoclast formation in calcitriol-stimulated mouse marrow cultures;

FIG. 6 is a graph showing that Binhib 2, which inhibits B subunit actin interactions in vitro, blocks ruffled membrane formation (right panel), without affecting osteoclast formation (left panel);

FIG. 7 depicts that enoxacin (10 μM) blocks ruffled membrane formation. Mature osteoclasts on bone slices were treated with enoxacin or vehicle control for 3 days, then fixed and labeled with an anti-E subunit antibody. Control osteoclasts produced many ruffled membranes, but in enoxacin-treated cultures, E subunit labeling was detected in a perinuclear distribution. Arrows point to representative osteoclasts;

FIG. 8 shows that miRNAs up and down-regulated in response to RANKL. Sample A is unstimulated, and sample B has been stimulated with RANKL for 5 Days;

FIG. 9 shows primary osteoclasts grown and stained for TRAP activity;

FIG. 10 depicts that example of pitting assay performed in the test. In this example, the effect of an extract from Brazilian propolis on bone resorption was evaluated;

FIG. 11 is a list of cytokines and chemokines to be tested using multiplex and 1 plex kits for Millipore. Screen makes use of Luminex xMAP apparatus;

FIG. 12 depicts the pathway that inhibition of IKK epsilon expression by increased miR-155 activity could lead to reduction of NF Kappa B signaling, inhibition of osteoclastogenesis and bone resorption.

FIG. 13 presents the effects of bis-enoxacin on mouse marrow cultures.

FIG. 14 depicts the effects on actin ring numbers through pre-treatment of bone slices with bis-enoxacin at concentrations of 10 mM and 1 mM. The pre-treatment reduced the actin ring numbers by approximately 60%.

FIG. 15 presents the effects of bis-enoxacin on bone resorption. The results demonstrate that each of the concentrations (30 mM, 10 mM, and 1 mM) of bis-enoxacin reduced bone resorption compared to control.

### DETAILED DESCRIPTION OF THE INVENTION

The invention relates to compounds for use in the treatment or prevention of an osteoclast-related disease or disorder in a subject identified as in need of the treatment of bone diseases, compositions and methods of use thereof. The compounds are capable of reducing bone resorption and stimulating new bone formation. In one embodiment, the compounds are capable of inhibiting (or interfering with) interactions between V-ATPase and F-actin in the membranes of osteoclasts.

Osteoclasts are cells that are dedicated to the resorption of bone (Teitelbaum, S. L. Science 2000, 289:1504-1 508). They utilize specialized membrane and cytoskeletal structures to create extracellular resorptive compartments between themselves and the bone into which they secrete protons and proteases that degrade the bone (Blair, H. C., et al. Science 1989, 245:855-857; Holliday, L. S., et al. J. Bioenerg. Biomembr. 2005, 37:419-423). Osteoclasts differentiate from hematopoetic precursors in response to stimulation by RANKL (Kong, Y. Y., et al. Nature 1999, 397:315-323; Burgess, T. L., et al. J. Cell Biol. 1999, 145:527-538). This process includes numerous changes in protein expression that are mediated at the transcriptional and post-transcriptional levels (Lee, B. S., et al. J. Bone Miner. Res. 1999, 14:21 27-21 36; Wang, S. P., et al. J. Biol. Chem. 2002, 277:8827-8834; Jeyaraj, S., et al. J. Biol. Chem. 2005, 280:37957-37964; Hiura, K., et al. Cell Motility and the Cytoskeleton 1995, 30:272-284; Hurst, I. R., et al. Journal of Bone and Mineral Research 2004, 19:499-506). Bone resorption by osteoclasts involves formation of unusual sub-compartments of the plasma membrane called ruffled membranes (or ruffled borders).

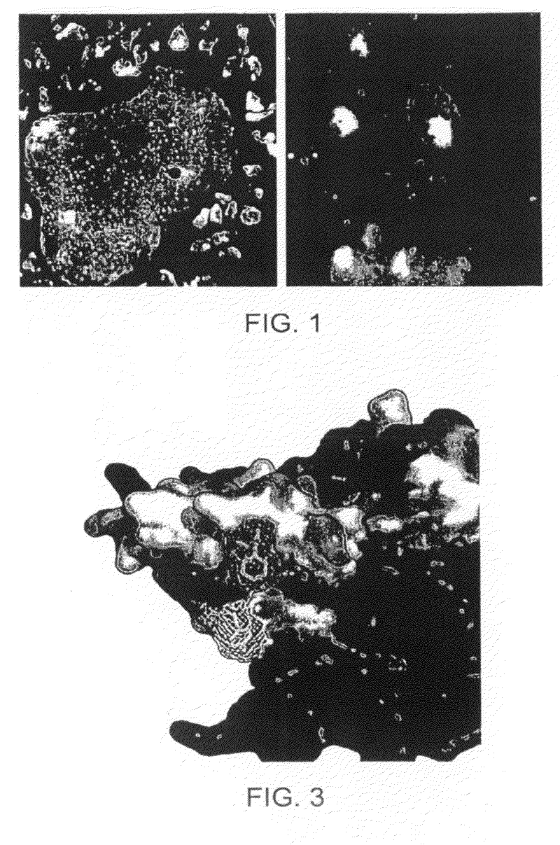

V-ATPases are enzymes that are forbidden entry into the plasma membrane in most cells-types, but pack the ruffled membrane (FIG. 1). The unusual transport of V-ATPases to ruffled membranes requires an actin binding site in the B subunit (Zuo, J., et al. J. Bone Miner. Res. 2006, 21:714-721). V-ATPases are expressed at low levels in most cell types and are excluded from the plasma membrane, but are present at very high levels in osteoclasts, where they are packed into the ruffled plasma membrane.

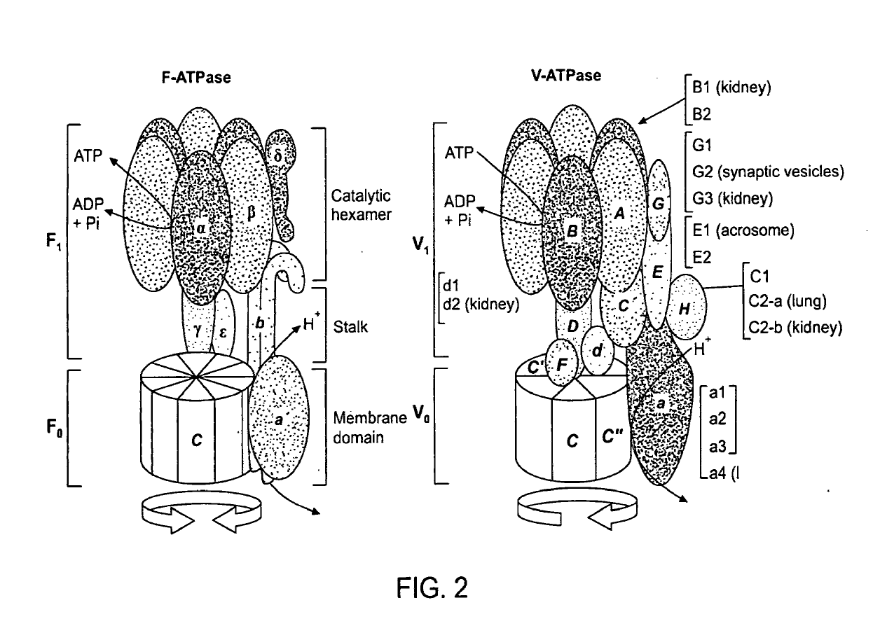

V-ATPases are composed of numerous subunits, some of which are present in multiple copies per holoenzyme (Gluck, S. L., et al. Acta Physiol Scand. Suppl 1998, 643:203-2 12) and are close relatives of the mitochondrial ATP synthase (FIG. 2). A number of subunits have isoforms that are tissue or cell type-specific. Subunits a1 and a2 are expressed ubiquitously in cells, including osteoclasts, and are found in endosomes and lysosomes. Subunit a3 is expressed at high levels in osteoclasts. Mutations in subunit a3 cause autosomal malignant osteoporosis (Ogbureke, K. U. E., et al. Frontiers in Bioscience 2005, 10:2940-U1 17).

The present inventors have now discovered a novel therapeutic strategy that treats or prevents osteoporosis or other osteoclast-related bone diseases or disorders through inhibition of interactions between V-ATPase and membranes of osteoclasts, in particular, through inhibiting (or interfering with) interaction between V-ATPase and F-actin in the membranes of osteoclasts.

Further, the present inventors have discovered unexpectedly small molecules are capable of reducing bone resorption and stimulating new bone formation. These molecules can act as anabolic therapeutic agents in treating or preventing osteoclast-related bone diseases or disorders in a subject. Without wishing to be bound by theory, in at least some cases, it is believed that these molecules are capable of interfering with interactions between V-ATPase and F-actin.

In another aspect, it has been also found recently that the unusual structure and function of osteoclasts is the product of altered patterns of gene expression. There are numerous unique elements to this gene expression pattern that are known. The specialized ability of osteoclasts to resorb bone is derived from altered patterns of gene expression that occur as osteoclasts differentiate from multipotent hematopoietic stem cells. Agents that alter these changes may prevent or reduce the formation of osteoclasts and hence lead to reductions in bone resorption.

The recent discovery of miRNAs provides an opportunity to explore unconventional therapeutic approaches for bone disease. MiRNAs have been identified as negative regulators of gene expression (Lau, N. C., et al. Science, 2001, 294:858-862; Lagos-Quintana, M., et al. Science, 2001, 294:853-858; Lee, R. C. et al. Science, 2001, 294:862-864). MiRNAs, which are produced endogenously in abundance and variety in mammalian cells, make use of elements of the RNA interference (RNAi) pathway to regulate the stability and expression of mRNAs (Murchison, E. P. et al. Curr. Opin. Cell Biol., 2004, 16:223-229; Almeida, R. et al. Trends Cell Biol. 2005, 15:251-258; Ambros, V. Nature, 2004, 431:350-355; Carthew, R. W. 2006. Gene regulation by microRNAs. Curr. Opin. Genet. Dev., 2006, 16:203-208). Further, miRNAs have already been shown to play regulatory roles in various cells and tissues. Although it is still very early in the study of miRNAs, it is certain that these small molecules play fundamental roles in regulating cellular metabolism, differentiation, and death in mammalian biology, and pathophysiology (Chiou, T. J., Plant Cell Environ. 2007, 30:323-332; Chen, C. Z. et al., Semin. Immunol., 2005, 17:155-165; Choong, M. L. et al. Exp. Hematol. 2007, 35:551-564; Dahm, R. et al. Semin. Cell Dev. Biol., 2007, Harfe, B. D. Curr. Opin. Genet. Dev. 2005, 15:41 0-41 5; Jovanovic, M. et al. Onco gene, 2006, 25:6176-6187; Matsubara, H., et al. Apoptosis induction by antisense oligonucleotides against miR-1 7-5p and miR-20a in lung cancers overexpressing miR-1 7-92. Onco gene., 2007; Bloomston, M., et al. JAMA, 2007, 297:1901-1908). Estimates have been made that as much as 30% of all genes may be regulated by miRNAs, and recent studies have already implicated miRNAs in various disease states, including cancer and congenital heart disease (see, for example, John, B., et al. PLoS. Biol. 2004, 2:e363).

However, to date there has been only one publication examining miRNAs in the regulation of osteoclasts, which reported that miRNA-223 (mmu-mir-223) is a key factor in osteoclast differentiation (Sugatani, T. et al. MicroRNA-223 is a key factor in osteoclast differentiation. J. Cell Biochem., 2007).

The present inventors have discovered that miRNA regulation as potentially vital for regulation of osteoclasts, and regulation of osteoclastogenesis by miRNAs provides opportunities for highly selective pharmaceutical manipulation of osteoclasts. Based on test data (see, e.g., the Examples, infra), the present inventors have found that osteoclasts likely rely on miRNA regulation heavily during the dramatic changes in gene expression that are required for their differentiation from multipotent stem cells.

Moreover, the present inventors have found that agents (such as enoxacin) that may manipulate miRNA expression or activity are a novel and viable class of osteoporosis drugs, and that methods of using those agents may prove to be a novel approach to the treatment of osteoclast-related or mediated disease or disorder.

Thus, the present invention also provides a method for treating or preventing an osteoclast-related disease or disorder in a subject identified as in need of the treatment of bone diseases. The method comprises administering to the subject an effective amount of a compound identified as capable of reducing bone resorption and stimulating new bone formation.

In one embodiment, the compound administered to the subject is capable of reducing bone resorption and stimulating new bone formation through inhibiting (or interfering with) interaction between V-ATPase and F-actin in the membranes of osteoclasts.

In certain embodiments, the compound is selected from the group of fluoroquinolone derivatives and bis-phosphonate derivatives, or a pharmaceutically acceptable salt, solvate, clathrate, hydrate, polymorph, or prodrug thereof. Certain embodiments provide that the compound is selected from the group of 1-ethyl-6-fluoro-4-oxo-7-(piperazin-1-yl)-1,4-dihydro-1,8-naphthyridine-3-carboxylic acid (“enoxacin”); 1-cyclopropyl-6-fluoro-4-oxo-7-piperazin-1-yl-quinoline-3-carboxylic acid (“ciprofloxacin”); 1-ethyl-6-fluoro-4-oxo-7-(piperazin-1-yl)-1,4-dihydroquinoline-3-carboxylic acid (“norfloxacin”); 1-ethyl-6-fluoro-7-(4-methylpiperazin-1-yl)-4-oxo-1,4-dihydroquinoline-3-carboxylic acid (“pefloxacin”); 3,5,7-triaza-1-azoniatricyclo[3.3.1.13,7]decane, 1-[2-(4-chlorophenyl)-2-oxoethyl]-, iodide (“Binhib 2”); and 7-(4-(2,2-diphosphonoethyl)piperazin-1-yl)-1-ethyl-6-fluoro-4-oxo-1,4-dihydro-1,8-naphthyridine-3-carboxylic acid (“bis-enoxacin”), or a pharmaceutically acceptable salt, solvate, clathrate, hydrate, polymorph, or prodrug thereof. In another embodiment, the compound is enoxacin or bis-enoxacin, or a pharmaceutically acceptable salt, solvate, clathrate, hydrate, polymorph, or prodrug thereof. In one embodiment, the compound is administered at a dosage other than which is normally required for the compound to demonstrate antibiotic activity (if applicable). Certain instances provide that the administration dosages of the compound in accordance with the invention are at levels lower than the dosages prescribed for therapeutically effective antibiotic activity.

In another embodiment, the compound is also capable of stimulating microRNA activity. One embodiment provides that the compound is enoxacin or bis-enoxacin, or a pharmaceutically acceptable salt, solvate, clathrate, hydrate, polymorph, or prodrug thereof.

Osteoclast-related diseases or disorders include, but are not limited to, periodontal disease, non-malignant bone disorders, osteoporosis, Paget's disease of bone, osteogenesis imperfecta, fibrous dysplasia, and primary hyperparathyroidism, estrogen deficiency, inflammatory bone loss, bone malignancy, arthritis, osteopetrosis, hypercalcemia of malignancy (HCM), osteolytic bone lesions of multiple myeloma and osteolytic bone metastases of breast cancer, and metastatic cancers, bone cancers, osteomyelitis, and osteoclast-related dental diseases or disorders.

In the case of overlap in these definitions, the disease, condition or disorder may be considered to be a member of any of the above listed classes of osteoclast-related diseases or disorders.

In one embodiment, the osteoclast-related disease or disorder is selected from the group of osteoporosis, bone cancers, and osteoclast-related dental diseases or disorders. In one embodiment, the osteoclast-related disease or disorder is a bone cancer selected from the group of osteosarcoma, Ewing sarcoma, malignant fibrous histiocytoma, and chondrosarcoma. In another embodiment, the osteoclast-related disease or disorder is a dental disease selected from the group of P. gingivalis infection, periodontal diseases, endodontic diseases/disorders and orthodontic tooth movement.

In another embodiment, the method further comprises treating a subject with an effective amount of an additional therapeutic agent selected from the group of bisphosphonates, calcitonin, intermittent PTH, Denosumib and mixtures thereof. In one embodiment, the compound of the invention and the additional therapeutic agent are administered simultaneously. Another embodiment provides that the compound and the additional therapeutic agent are administered sequentially.

Another aspect of the invention provides a method for identifying a compound for treating or preventing an osteoclast-related disease or disorder in a subject identified as in need of a treatment of bone diseases, the method comprising a) designing computer-based screen to identify a compound that is membrane-permeable, wherein the compound is also capable of binding the actin binding site of V-ATPase; and b) testing the compound for its ability to inhibit (or interfere with) interaction between recombinant V-ATPase and F-actin in vitro or in vivo. In one embodiment, the method uses pure recombinant V-ATPase. In another embodiment, the method uses pure F-actin.

In one embodiment, the method further comprises assessing the compound for its ability to disrupt osteoclast bone resorption in vitro cell culture. In another embodiment, the method also includes testing the compound for effects on bone loss in vivo. In one embodiment, the testing in vivo uses ovariectomized rats.

Further aspects provide that compounds identified herein are capable of reducing bone resorption and stimulating new bone formation. In one embodiment, the compound is capable of reducing bone resorption and stimulating new bone formation through inhibiting (or interfering with) interaction between V-ATPase and F-actin in the membranes of osteoclasts.

In another embodiment, the compounds identified herein are capable of stimulating miRNA activity.

In another aspect, the invention provides a kit for treating or preventing an osteoclast-related disease or disorder in a subject identified as in need of a treatment of bone diseases. The kit includes a compound herein, or its pharmaceutically acceptable salt, solvate, clathrate, hydrate, polymorph, or prodrug thereof, and instructions for use. In certain embodiments, the invention provides: a kit comprises a compound capable of reducing bone resorption and stimulating new bone formation. In one embodiment, the compound acts through inhibiting (or interfering with) interaction between V-ATPase and F-actin in the membranes of osteoclasts.

Another aspect of the invention provides a method for treating or preventing an osteoclast-related disease or disorder in a subject identified as in need of the treatment of bone diseases, wherein the method includes a) identifying or selecting a subject suffering from or susceptible to an osteoclast-related bone disease for treatment with a compound identified as capable of reducing bone resorption and stimulating new bone formation; b) monitoring the progress of the subject being treated; and c) assessing the efficacy of the treatment in the subject. Identifying or selecting a subject in need can be in the judgment of a subject or a health care professional and can be subjective (e.g. opinion) or objective (e.g. measurable by a test or diagnostic method).

In another aspect, the invention provides a packaged composition including an effective amount of a compound identified as capable of reducing bone resorption and stimulating new bone formation, and a pharmaceutically acceptable carrier or diluent. In one embodiment, the compound acts through inhibiting (or interfering with) interaction between V-ATPase and F-actin in the membranes of osteoclasts. The composition may be formulated for treating a subject suffered from or susceptible to an osteoclast-related bone disease or disorder, and packaged with instructions to treat a subject with such a need.

Another aspect of the present invention provides a pharmaceutical composition for treating or preventing an osteoclast-related disease or disorder in a subject identified as in need of the treatment of bone diseases. The composition includes an effective amount of a compound identified as capable of reducing bone resorption and stimulating new bone formation, and a pharmaceutically acceptable carrier. In one embodiment, the compound is capable of reducing bone resorption and stimulating new bone formation through inhibiting (or interfering with) interaction between V-ATPase and F-actin in the membranes of osteoclasts.

In one embodiment, the compound is selected from the group of 1-ethyl-6-fluoro-4-oxo-7-(piperazin-1-yl)-1,4-dihydro-1,8-naphthyridine-3-carboxylic acid (“enoxacin”); 1-cyclopropyl-6-fluoro-4-oxo-7-piperazin-1-yl-quinoline-3-carboxylic acid (“ciprofloxacin”); 1-ethyl-6-fluoro-4-oxo-7-(piperazin-1-yl)-1,4-dihydroquinoline-3-carboxylic acid (“norfloxacin”); 1-ethyl-6-fluoro-7-(4-methylpiperazin-1-yl)-4-oxo-1,4-dihydroquinoline-3-carboxylic acid (“pefloxacin”); 3,5,7-triaza-1-azoniatricyclo[3.3.1.13,7]decane, 1-[2-(4-chlorophenyl)-2-oxoethyl]-, iodide (“Binhib 2”); and 7-(4-(2,2-diphosphonoethyl)piperazin-1-yl)-1-ethyl-6-fluoro-4-oxo-1,4-dihydro-1,8-naphthyridine-3-carboxylic acid (“bis-enoxacin”), or a pharmaceutically acceptable salt, solvate, clathrate, hydrate, polymorph, or prodrug thereof. In another embodiment, the compound is enoxacin or bis-enoxacin, or a pharmaceutically acceptable salt, solvate, clathrate, hydrate, polymorph, or prodrug thereof.

In one embodiment, the compound is also capable of stimulating microRNA activity. In one embodiment, the compound is enoxacin or bis-enoxacin, or its pharmaceutically acceptable salt, solvate, clathrate, hydrate, polymorph, or prodrug thereof.

The invention also provides methods for designing, evaluating and identifying a compound that is capable of reducing bone resorption and stimulating new bone formation.

### 1. DEFINITIONS

Before further description of the invention, and in order that the invention may be more readily understood, certain terms are first defined and collected here for convenience.

As used in the specification and claims, the singular term “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally effect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The term “agent” is meant a small molecule compound, a polypeptide, polynucleotide, or fragment, or analog thereof, or other biologically active molecule.

The term “anabolic” refers to a process producing growth and differentiation of cells and increase organs and tissues in a subject. Examples of anabolic processes include new bone formation or growth, and increases in muscle mass.

The term “associating with” refers to a condition of proximity between a chemical entity or compound, or portions thereof, and a binding pocket or binding site on a protein. The association may be non-covalent (wherein the juxtaposition is energetically favored by hydrogen bonding or van der Waals or electrostatic interactions) or it may be covalent.

The term “binding pocket”, as used herein, refers to a region of a molecule or molecular complex, that, as a result of its shape, favorably associates with another chemical entity or compound.

The language “biological activities” of a compound of the invention includes all activities elicited by compound of the inventions in a responsive cell. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In one embodiment, a suitable biological composition or biological sample is a red blood cell suspension. In some embodiments, the blood cell suspension includes mammalian blood cells. In certain instances, the blood cells are obtained from a human, a non-human primate, a dog, a cat, a horse, a cow, a goat, a sheep or a pig. In certain embodiments, the blood cell suspension includes red blood cells and/or platelets and/or leukocytes and/or bone marrow cells.

The term “bone cancers” generally refer to cancers that form in any type of bone tissue. Primary bone cancer is cancer that forms in cells of the bone. Common types of primary bone cancer include, but are not limited to, osteosarcoma, Ewing sarcoma, malignant fibrous histiocytoma, and chondrosarcoma. Secondary bone cancer is cancer that spreads to the bone from another part of the body (such as the prostate, breast, or lung).

The term “chiral” refers to molecules which have the property of non-superimposability of the mirror image partner, while the term “achiral” refers to molecules which are superimposable on their mirror image partner.

The term “clathrate” means a compound of the present invention or a salt thereof in the form of a crystal lattice that contains spaces (e.g., channels) that have a guest molecule (e.g., a solvent or water) trapped within.

The terms “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “dental diseases or disorders” refer to any disease, disorder or symptoms thereof that are affect the teeth or gums in a subject. In particular, the term in the present disclosure includes, but not limited to, gingivitis, periodontitis, dental erosion, cracked tooth syndrome, temporomandibular joint disorder, dental caries, xerostomia, orthodontic problems, problems needing endodontic treatments.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient to treat a disorder delineated herein. An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 30 mg/kg body weight. Certain ranges are about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In one example, a subject is treated with a compound of the invention in the range of between about 0.1 to 20 mg/kg body weight, one time per week for between about 1 to 10 weeks, between 2 to 8 weeks, between about 3 to 7 weeks, or for about 4, 5, or 6 weeks. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “homeostasis” is art-recognized to mean maintenance of static, or constant, conditions in an internal environment.

The term “hydrate” means a compound of the present invention or a salt thereof, which further includes a stoichiometric or non-stoichiometric amount of water bound by non-covalent intermolecular forces.

The language “improved biological properties” refers to any activity inherent in a compound of the invention that enhances its effectiveness in vivo. In one embodiment, this term refers to any qualitative or quantitative improved therapeutic property of a compound of the invention, such as reduced toxicity.

The term “in combination with” is intended to refer to all forms of administration that provide an a compound of the invention together with an additional pharmaceutical agent, such as a second compound used in clinic for treating or preventing osteoclast-related disease or disorder, where the two are administered concurrently or sequentially in any order.

The term “osteoclast-related” includes any disease, disorder or symptoms thereof that are mediated by activities of osteoclasts. The term includes, but is not limited to, periodontal disease, non-malignant bone disorders, osteoporosis, Paget's disease of bone, osteogenesis imperfecta, fibrous dysplasia, and primary hyperparathyroidism, estrogen deficiency, inflammatory bone loss, bone malignancy, arthritis, osteopetrosis, hypercalcemia of malignancy (HCM), osteolytic bone lesions of multiple myeloma and osteolytic bone metastases of breast cancer, and metastatic cancers, bone cancers, and dental diseases or disorders. In the case of overlap in these definitions, the disease, condition or disorder may be considered to be a member of any of the above listed classes of osteoclast-related” diseases or disorders.

The term “isomers” or “stereoisomers” refers to compounds which have identical chemical constitution, but differ with regard to the arrangement of the atoms or groups in space.

The term “modulate” refers to an increase or decrease, e.g., in the ability of a compound inhibit activity of a target in response to exposure to a compound of the invention, including for example in an subject (e.g., animal, human) such that a desired end result is achieved, e.g., a therapeutic result.

The term “obtaining” as in “obtaining a compound capable of modulating (agonizing, antagonizing) a target delineated herein and is intended to include purchasing, synthesizing or otherwise acquiring the compound.

The term “pharmaceutically acceptable salt,” is a salt formed from, for example, an acid and a basic group of a compound of any one of the formulae disclosed herein. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “a pharmaceutically acceptable salt” also refers to a salt prepared from a compound of any one of the formulae disclosed herein having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of the formulae disclosed herein having a basic functional group, such as an amino functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

The term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the invention or otherwise described herein which is effective, upon single or multiple dose administration to the patient, in preventing or treating a disorder herein.

The language “reduced toxicity” is intended to include a reduction in any undesired side effect elicited by a compound of the invention when administered in vivo.

The term “subject” includes organisms which are capable of suffering from a disease or disorder herein or who could otherwise benefit from the administration of a compound of the invention, such as human and non-human animals. Preferred humans include human patients suffering from or prone to suffering from osteoclast-related bone diseases, disorders, or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The language “therapeutically effective amount” of a compound of the invention refers to an amount of an agent which is effective, upon single or multiple dose administration to the patient, treating or preventing osteoporosis and/or osteroclast-related symptoms, or in prolonging the survivability of the patient with such an osteoporosis beyond that expected in the absence of such treatment.

With respect to the nomenclature of a chiral center, terms “d” and “1” configuration are as defined by the IUPAC Recommendations. As to the use of the terms, diastereomer, racemate, epimer and enantiomer is used in their normal context to describe the stereochemistry of preparations.

### 2. COMPOUNDS OF THE INVENTION

In one aspect, the invention provides compounds identified as therapeutic agents for treating or preventing an osteoclast-related disease or disorder in a subject identified as in need of the treatment of bone diseases. The compounds herein are capable of reducing bone resorption and stimulating new bone formation.

One embodiment provides that a compound of the invention is identified as an inhibitor of interaction between vacuolar H⁺ ATPase (V-ATPase) and F-actin in the membranes of osteoclasts.

In another embodiment, the compound is capable of reducing bone resorption and stimulating new bone formation through inhibiting (or interfering with) interaction between V-ATPase and F-actin in the membranes of osteoclasts.

In another embodiment, the compound is a fluoroquinolone derivative or bis-phosphonate derivative, or a pharmaceutically acceptable salt, solvate, clathrate, hydrate, polymorph, or prodrug thereof.

In another aspect, the invention provides compounds as therapeutic agents for treating or preventing an osteoclast-related disease or disorder in a subject identified as in need of the treatment of bone diseases, wherein the compounds are capable of reducing bone resorption, stimulating new bone formation, and stimulating microRNA activity

Certain compounds of the invention include compounds specifically delineated herein:

### 1) 1-Ethyl-6-fluoro-4-oxo-7-(piperazin-1-yl)-1,4-dihydro-1,8-naphthyridine-3-carboxylic acid (“enoxacin”)

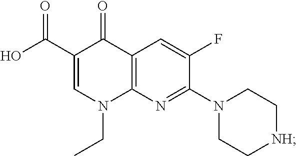

### 2) 1-Cyclopropyl-6-fluoro-4-oxo-7-piperazin-1-yl-quinoline-3-carboxylic acid (“ciprofloxacin”)

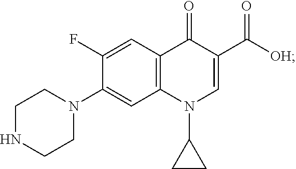

### 3) 1-Ethyl-6-fluoro-4-oxo-7-(piperazin-1-yl)-1,4-dihydroquinoline-3-carboxylic acid (“norfloxacin”)

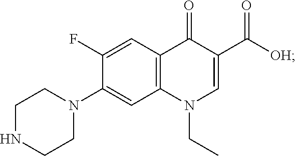

### 4) 1-Ethyl-6-fluoro-7-(4-methylpiperazin-1-yl)-4-oxo-1,4-dihydroquinoline-3-carboxylic acid (“pefloxacin”)

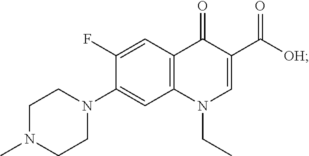

### 5) 3,5,7-Triaza-1-azoniatricyclo[3.3.1.13,7]decane, 1-[2-(4-chlorophenyl)-2-oxoethyl]-, iodide (“Binhib 2”)

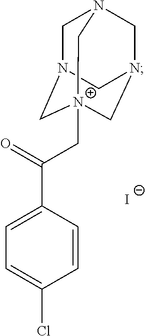

and

### 6) 7-(4-(2,2-Diphosphonoethyl)piperazin-1-yl)-1-ethyl-6-fluoro-4-oxo-1,4-dihydro-1,8-naphthyridine-3-carboxylic acid (“bis-enoxacin”)

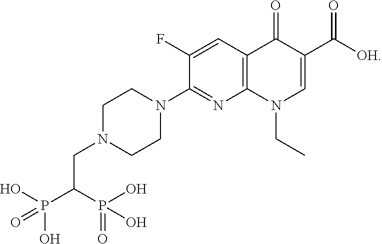

In one aspect, the compounds of the invention can also be bis-phosphonate derivatives. The invention also relates to a pharmaceutically acceptable salt, solvate, clathrate, hydrate, polymorph, or prodrug thereof, of the compounds mentioned infra.

The compounds of the invention may contain one or more asymmetric centers and thus occur as racemates and racemic mixtures, single enantiomers, individual diastereomers and diastereomeric mixtures. All such isomeric forms of these compounds are expressly included in the invention. The compounds of the invention may also be represented in multiple tautomeric forms, in such instances, the invention expressly includes all tautomeric forms of the compounds described herein. All such isomeric forms of such compounds are expressly included in the present invention. All crystal forms of the compounds described herein are expressly included in the invention.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

In yet another aspect, the invention provides the use of a compound of the invention, alone or together with one or more additional therapeutic agents in the manufacture of a medicament, either as a single composition or as separate dosage forms, for treatment or prevention in a subject of a disease, disorder or symptom set forth herein. Another aspect of the invention is a compound of the invention for use in the treatment or prevention in a subject of a disease, disorder or symptom thereof delineated herein.

Methods of synthesizing compounds herein are within the means of chemists of ordinary skill in the art (see, e.g., Herczegh et al., Osteoadsorptive Bisphosphonate Derivatives of Fluoroquinolone Antibacterials, J. Med. Chem. 2002, 45, 2338-2341). Methods for optimizing reaction conditions, if necessary minimizing competing by-products, are known in the art. The methods may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

### 3. METHODS AND USES RELATED TO THE COMPOUNDS OF THE INVENTION

In one aspect, the invention provides methods for treating or preventing an osteoclast-related disease or disorder in a subject identified as in need of the treatment of bone diseases, by administering to the subject an effective amount of a compound identified as capable of reducing bone resorption and stimulating new bone formation. Certain embodiments provide that the compound is capable of reducing bone resorption and stimulating new bone formation through inhibiting (or interfering with) interaction between V-ATPase and F-actin in the membranes of osteoclasts.

In certain embodiments, a compound of the invention is administered at a dosage other than a dosage that is normally required for the compound to show antibiotic effects in a subject. One embodiment provides that the administration dosage of the compound in accordance with the invention is lower than that is required for the compound to show antibiotic activity. Determination of an antibiotic dosage of a specific compound can be readily made by the physician or veterinarian (the “attending clinician”), as one skilled in the art, by the use of known techniques or by following medical protocols.

In certain embodiments, the subject has not been diagnosed as in a need for a treatment with antibiotics. In another embodiment, the subject is identified by an attending physician or veterinarian as not being in need of a treatment with antibiotics.

In another aspect, the methods herein include: wherein a compound identified as useful in reducing bone resorption is administered to a subject identified as in need of a treatment of bone diseases; wherein a compound identified as useful in stimulating new bone formation is administered to the subject; or wherein a compound identified as capable of inhibiting (or interfering with) interaction between V-ATPase and F-actin in the membranes of osteoclasts is administered to the subject.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method). In other methods, the subject is prescreened or identified as in need of such treatment by assessment for a relevant marker or indicator of suitability for such treatment.

In certain embodiments, the compound of the invention can be used in combination therapy with existing drugs to treat an osteoclast-related bone disease or disorder. These existing drugs are including, but not limited to, bisphosphonates, calcitonin, intermittent PTH, denosumib, teriparatide and strontium ranelate.

In certain embodiments, the methods of the invention include administering to a subject with an osteoclast-related bone disease or disorder a therapeutically effective amount of a compound of the invention in combination with another pharmaceutically active compound. Examples of pharmaceutically active compounds include compounds known to treat an osteoclast-related disease, e.g., existing anti-osteoporosis agents, antitumor agents, antiangiogenesis agents, chemotherapeutics, antibodies, etc. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 62th Edition 2008, Oradell N.J., Medical Economics Co., the complete contents of which are expressly incorporated herein by reference. The compound of the invention and the pharmaceutically active compound may be administered to the patient in the same pharmaceutical composition or in different pharmaceutical compositions (at the same time or at different times).

Determination of a therapeutically effective amount or a prophylactically effective amount of the compound of the invention can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific disease or disorder involved; pharmacodynamic characteristics of the particular agent and its mode and route of administration; the desired time course of treatment; the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective amount of a compound of the invention is expected to vary from about 0.1 milligram per kilogram of body weight per day (mg/kg/day) to about 100 mg/kg/day.

Compounds determined to be effective for the prevention or treatment of an osteoclast-related disease or disorder in a subject, e.g., dogs, chickens, and rodents, may also be useful in treatment of tumors in humans. Those skilled in the art of treating tumors in humans will know, based upon the data obtained in animal studies, the dosage and route of administration of the compound to humans.

The identification of a subject as in need for a treatment of bone diseases is well within the ability and knowledge of one skilled in the art. Certain of the methods for identification of a subject as in need for a treatment of bone diseases or who are at risk of developing such a disease/disorder which can be treated by the subject method are appreciated in the medical arts, such as family history, and the presence of risk factors associated with the development of that disease state in the patient. A clinician skilled in the art can readily identify such candidate patients, by the use of, for example, clinical tests, physical examination and medical/family history.

A method of assessing the efficacy of a treatment in a subject includes determining the pre-treatment extent of an osteoclast-related disease/disorder by methods well known in the art and then administering to the subject a therapeutically effective amount of a compound capable of reducing bone resorption and stimulating new bone formation in accordance with the invention. After an appropriate period of time after the administration of the compound (e.g., 1 day, 1 week, 2 weeks, one month, six months), the extent and the severity of the disease/disorder are determined again. The modulation (e.g., decrease) of the extent or severity of the an osteoclast-related bone disease or disorder indicates efficacy of the treatment. The extent or severity of the an osteoclast-related bone disease or disorder may be determined periodically throughout treatment. For example, the extent or severity of the an osteoclast-related bone disease or disorder may be checked every few hours, days or weeks to assess the further efficacy of the treatment. A decrease in extent or severity of an osteoclast-related bone disease or disorder indicates that the treatment is efficacious. The method described may be used to screen or select patients that may benefit from treatment with a compound described herein.

Yet another aspect provides a method to identify a compound for treating or preventing an osteoclast-related bone disease or disorder in a subject, wherein the compound is an inhibitor on the interaction between V-ATPase and F-actin. The method includes a) identifying structural pockets in actin-binding site of V-ATPase for interaction with the compound through using a computer-based screen; b) predicting interactions between said compound and said structural pockets by using docking simulations; and c) evaluating inhibitory activity of the compound against actin-binding site of V-ATPase by using a pelleting assay.

In one embodiment, the method may also include obtaining the crystal structure of the α subunit of the V-ATPase, or specific domains thereof (optionally apo form or complexed) or generating an atomic-level model of the B2 subunit, or specific domains thereof (optionally apo form or complexed), in the presence and/or absence of the test compound.

The method may also use information generated by analysis of crystal structures of the profilin 1-actin complex. Because part of the actin binding region of subunit B2 shares sequence homology with the actin binding site of profilin 1. In one embodiment, the methods may also use information from analysis of single amino acid substitutions in the subunit B2 actin binding domain.

In one embodiment, X-ray crystallography is utilized to characterize the mechanism of action for selected inhibitors to identify specific structural pocket for the subunit B2 actin binding domain. Then, compounds are computer-screened for their abilities to interact with this structural pocket on the subunit B2 actin binding domain based on the coordinates derived from the crystal structure.

In another aspect, a compound of the invention is packaged in a therapeutically effective amount with a pharmaceutically acceptable carrier or diluent. The composition may be formulated for treating or preventing an osteoclast-related bone disease or disorder in a subject identified as in need of the treatment of bone diseases, and packaged with instructions to treat the subject suffering from or susceptible to an osteoclast-related bone disease/disorder.

In another aspect, methods of treating or preventing an osteoclast-related disease or disorder include administering an effective amount of a compound of the invention (i.e., a compound described herein) to a subject identified as in need of the treatment of bone diseases. The administration may be performed by any route of administering known in the pharmaceutical arts.

If the modulation of the status indicates that the subject may have a favorable clinical response to the treatment, the subject may be treated with the compound of the invention. For example, the subject can be administered therapeutically effective dose or doses of the compound.

Kits of the invention include kits for treating or preventing an osteoclast-related disease or disorder in a subject identified as in need of the treatment of bone diseases. The kit may include a compound of the invention, for example, a compound described herein, pharmaceutically acceptable esters, salts, and prodrugs thereof, and instructions for use. The instructions for use may include information on dosage, method of delivery, storage of the kit, etc. The kits may also include, reagents, for example, test compounds, buffers, media (e.g., cell growth media), cells, etc. Test compounds may include known compounds or newly discovered compounds, for example, combinatorial libraries of compounds. One or more of the kits of the invention may be packaged together, for example, a kit for assessing the efficacy of an osteoclast-related disease treatment may be packaged with a kit for monitoring the progress of a subject being treated according to the invention.

In another aspect, the compound included in the kits can also be those: wherein the compound is identified as useful in'reducing bone resorption for administration to the subject; wherein the compound is identified as useful in stimulating new bone formation for administration to the subject; or wherein the compound is identified to inhibit interaction between V-ATPase and F-actin in the membranes of osteoclasts for administration to the subject.

The present methods can be performed on cells in culture, e.g. in vitro or ex vivo, or on cells present in an animal subject, e.g., in vivo. Compounds of the invention can be initially tested in vitro using primary cultures of proliferating cells, e.g., transformed cells, tumor cell lines, and the like. Compound of the invention can be initially tested in vitro using cells or other mammalian or non-mammalian animal models. Alternatively, the effects of compound of the invention can be characterized in vivo using animals models.

Another aspect is the use of a compound of the invention in the manufacture of a medicament for treating or preventing an osteoclast-related disease or disorder in a subject identified as in need of a treatment of bone diseases. One embodiment provides that the medicament is used for treatment or prevention in a subject of a disease, disorder or symptom set forth above.

### 4. PHARMACEUTICAL COMPOSITIONS

The invention also provides a pharmaceutical composition, comprising an effective amount of a compound of the present invention and a pharmaceutically acceptable carrier. In a further embodiment, the effective amount is effective for treating or preventing an osteoclast-related disease or disorder in a subject identified as in need of a treatment of bone diseases, as described previously.

In an embodiment, the compound of the invention is administered to a subject with a need using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, more preferably from about 10 percent to about 30 percent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (or 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the compound of the invention is the maximum that a patient can tolerate and not develop serious side effects. In certain instances, the compound of the invention is administered at a concentration of about 0.001 mg to about 100 mg per kilogram of body weight, about 0.001-about 10 mg/kg or about 0.001 mg-about 100 mg/kg of body weight. Ranges intermediate to the above-recited values are also intended to be part of the invention.

### 5. SCREENING METHODS AND SYSTEMS

In another aspect, the invention provides a machine readable storage medium which comprises the structural coordinates of either one or both of the binding pockets identified herein, or similarly shaped, homologous binding pockets. Such storage medium encoded with these data are capable of displaying a three-dimensional graphical representation of a molecule or molecular complex which comprises such binding pockets on a computer screen or similar viewing device.

The invention also provides methods for designing, evaluating and identifying a compound which is capable of reducing bone resorption and stimulating new bone formation. In one embodiment, the compound is capable of reducing bone resorption through inhibiting (or interfering with) interaction between V-ATPase and F-actin in the membranes of osteoclasts. Thus, the computer produces a three-dimensional graphical structure of a molecule or a molecular complex which comprises a binding pocket.

In another embodiment, the invention provides a computer for producing a three-dimensional representation of a molecule or molecular complex defined by structure coordinates of Subunit B2 of V-ATPase or domains thereof, or a three-dimensional representation of a homologue of said molecule or molecular complex, wherein said homologue comprises a binding pocket that has a root mean square deviation from the backbone atoms of said amino acids of not more than 2.0 (more preferably not more than 1.5) angstroms.

In exemplary embodiments, the computer or computer system can include components which are conventional in the art, e.g., as disclosed in U.S. Pat. Nos. 5,978,740 and/or 6,183,121 (incorporated herein by reference). For example, a computer system can includes a computer comprising a central processing unit (“CPU”), a working memory (which may be, e.g., RAM (random-access memory) or “core” memory), a mass storage memory (such as one or more disk drives or CD-ROM drives), one or more cathode-ray tube (CRT) or liquid crystal display (LCD) display terminals, one or more keyboards, one or more input lines, and one or more output lines, all of which are interconnected by a conventional system bus.

Machine-readable data of this invention may be inputted to the computer via the use of a modem or modems connected by a data line. Alternatively or additionally, the input hardware may include CD-ROM drives, disk drives or flash memory. In conjunction with a display terminal, a keyboard may also be used as an input device.

Output hardware coupled to the computer by output lines may similarly be implemented by conventional devices. By way of example, output hardware may include a CRT or LCD display terminal for displaying a graphical representation of a binding pocket of this invention using a program such as QUANTA or PYMOL. Output hardware might also include a printer, or a disk drive to store system output for later use.

In operation, the CPU coordinates the use of the various input and output devices, coordinates data accesses from the mass storage and accesses to and from working memory, and determines the sequence of data processing steps. A number of programs may be used to process the machine-readable data of this invention, including commercially-available software.

A magnetic storage medium for storing machine-readable data according to the invention can be conventional. A magnetic data storage medium can be encoded with a machine-readable data that can be carried out by a system such as the computer system described above. The medium can be a conventional floppy diskette or hard disk, having a suitable substrate which may be conventional, and a suitable coating, which may also be conventional, on one or both sides, containing magnetic domains whose polarity or orientation can be altered magnetically. The medium may also have an opening (not shown) for receiving the spindle of a disk drive or other data storage device.

The magnetic domains of the medium are polarized or oriented so as to encode in manner which may be conventional, machine readable data such as that described herein, for execution by a system such as the computer system described herein.

An optically-readable data storage medium also can be encoded with machine-readable data, or a set of instructions, which can be carried out by a computer system. The medium can be a conventional compact disk read only memory (CD-ROM) or a rewritable medium such as a magneto-optical disk which is optically readable and magneto-optically writable.

In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating.

In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with the structural pocket of human thymidylate synthase which in turn inhibits interaction between V-ATPase and F-actin in the membranes of osteoclasts are potential drug candidates. Alternatively, the structure encoded by the data may be displayed in a graphical three-dimensional representation on a computer screen. This allows visual inspection of the structure, as well as visual inspection of the structural pocket's association with chemical entities.

Thus, according to another embodiment, the invention relates to a method for evaluating the potential of a chemical entity to associate with a) a molecule or molecular complex of the B2 subunit of V-ATPase, or specific domains thereof, or b) a homologue of said molecule or molecular complex, wherein said homologue comprises a structural pocket that has a root mean square deviation from the backbone atoms of said amino acids of not more than 2.0 (more preferably 1.5) angstroms.

This method comprises the steps of:

i) employing computational means to perform a fitting operation between the chemical entity and structural pockets of Subunit B2 actin binding site or a complex thereof; and

ii) analyzing the results of the fitting operation to quantify the association between the chemical entity and the structural pockets. The term “chemical entity”, as used herein, refers to a chemical compound, complexes of at least two chemical compounds, and fragments of such compounds or complexes.

One embodiment uses crystallized α subunit of F-ATPase. In another embodiment, 3D-PSSM and Swiss Model are used to perform comparative structural modeling to generate an atomic-level model of the B2 subunit.

In one embodiment, the invention provides an approach utilized: (1) generation of a multiple alignment with the sequence to be modeled, (2) generation of a framework for the new sequence based on superposition of the related three-dimensional structure, (3) rebuilding loops and side chains, (4) structural refinement by energy minimization and molecular dynamics, and (5) verification of the model structure geometry.

The design of compounds capable of reducing bone resorption and stimulating new bone formation. In one embodiment, it is believed that the compounds capable of reducing bone resorption and stimulating new bone formation act through inhibiting (or interfering with) interaction between V-ATPase and F-actin in the membranes of osteoclasts, generally involves consideration of several factors. First, the chemical entity must be capable of physically and structurally associating with parts or all of the binding site in the B2 subunit of V-ATPase. Non-covalent molecular interactions important in this association include hydrogen bonding, van der Waals interactions, hydrophobic interactions and electrostatic interactions. Second, the entity must be able to assume a conformation that allows it to associate with the structural pocket of the B2 subunit of the V-ATPase directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the binding pocket or homologues thereof.

The potential inhibitory or binding effect of a chemical entity on the B2 subunit of the V-ATPase or specific domains thereof-related binding pocket may be analyzed prior to its actual synthesis and testing by the use of computer modeling techniques. If the theoretical structure of the given entity suggests insufficient interaction and association between it and the target binding pocket, testing of the entity is obviated. However, if computer modeling indicates a strong interaction, the molecule may then be synthesized and tested for its ability to bind to a binding pocket. This may be achieved, e.g., by testing the ability of the molecule to bind B2 subunit of the V-ATPase, or specific domains thereof activity, e.g., using assays described herein or known in the art. In this manner, synthesis of inoperative compounds may be avoided.

A potential inhibitor of the B2 subunit of the V-ATPase or, or specific domains thereof-related structural pocket may be computationally evaluated by means of a series of steps in which chemical entities or fragments are screened and selected for their ability to bind the B2 subunit of the V-ATPase, or specific domains thereof-related binding pockets.

One skilled in the art may use one of several methods to screen chemical entities or fragments for their ability to bind the B2 subunit of the V-ATPase, or specific domains thereof-related structural pocket. This process may begin by visual inspection of, for example, the B2 subunit of the V-ATPase, or specific domains thereof-related binding pocket on the computer screen based on binding site of the B2 subunit of the V-ATPase, or specific domains thereof structure coordinates described herein, or other coordinates which define a similar shape generated from the machine-readable storage medium. Selected fragments or chemical entities may then be positioned in a variety of orientations, or docked, within that binding pocket as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding pocket.

Instead of proceeding to build an inhibitor of a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed in accordance with this invention [see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994).

Once a compound has been designed or selected, the efficiency with which that entity may bind to a binding pocket may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages are known to those skilled in the art.

Another technique involves the in silico screening of virtual libraries of compounds, e.g., as described herein. Many thousands of compounds can be rapidly screened and the best virtual compounds can be selected for further screening (e.g., by synthesis and in vitro testing). Small molecule databases can be screened for inhibitory activities against interaction between V-ATPase and F-actin in the membranes of osteoclasts, or binding to the structural pocket of the B2 subunit of the V-ATPase. In this screening, the quality of fit of such entities to the binding site may be judged either by shape complementarity or by estimated interaction energy.

### 6. KITS

The invention also provides kits. The kits include an effective amount of a compound that is capable of reducing bone resorption and stimulating new bone formation, together with instructions for use of the kit. In particular, the kit includes a compound that is capable of reducing bone resorption and stimulating new bone formation through inhibiting or interfering with interaction between V-ATPase and F-actin in the membranes of osteoclasts.

The kits also include instructions for use in treating or preventing an osteoclast-related disease or disorder in a subject. The osteoclast-related diseases or disorders include, but not limited to, periodontal disease, non-malignant bone disorders, osteoporosis, Paget's disease of bone, osteogenesis imperfecta, fibrous dysplasia, and primary hyperparathyroidism, estrogen deficiency, inflammatory bone loss, bone malignancy, arthritis, osteoporosis, hypercalcemia of malignancy (HCM), osteolytic bone lesions of multiple myeloma and osteolytic bone metastases of breast cancer, and metastatic cancers, bone cancers, and osteoclast-related dental diseases or disorders.

The term “carrier” means delivery systems that are suited for containing one or more container means, such as, vials, tubes, and the like, whereas each of the container means comprising one of the separate elements to be used in the method. In view of the description provided herein of invention methods, those of skill in the art can readily determine the apportionment of the necessary reagents among the container means.

### EXAMPLES

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### Example 1

### Database of Small Molecules

The NCI/DTP maintains a repository of approximately 240,000 samples (i.e., the plated compound set) which are non-proprietary and offered to the research community for discovery and development of new agents for the treatment of cancer, AIDS, or opportunistic infections afflicting subjects with cancer or AIDS. The three-dimensional coordinates for the NCI/DTP plated compound set is obtained in the MDL SD format (www.chm.tu-dresden.de/edv/vamp65/REFERS/vr_{—}03d.htm) and converted to the mol2 format by the DOCK utility program SDF2MOL2. Partial atomic charges, solvation energies and van der Waals parameters for the ligands are calculated using SYBDB and added to the plated compound set mol2 files.

### Example 2

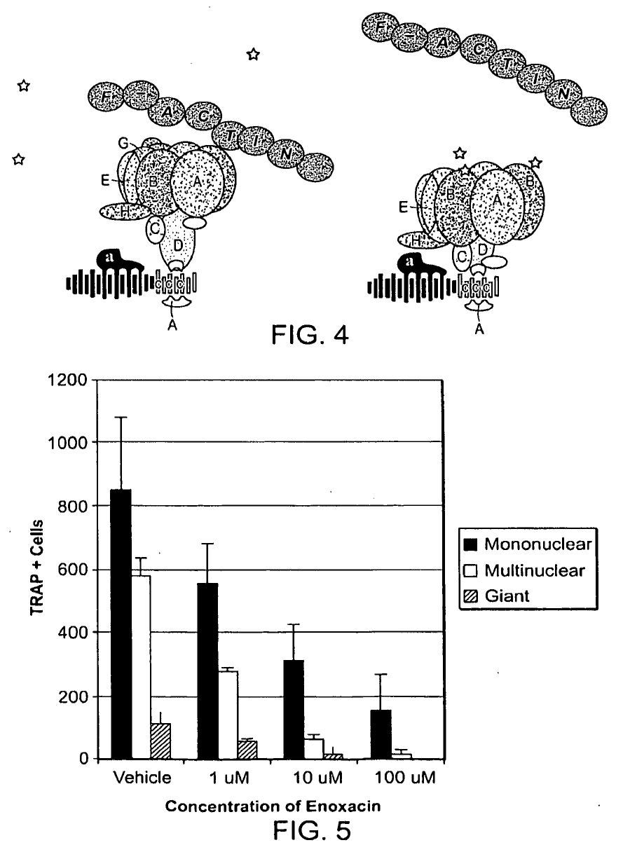

Screening for small molecule inhibitors of V-ATPase B subunit interaction: To identify small molecules that block the interaction between V-ATPase and F-actin in osteoclasts (FIG. 4), the present inventors screened for small, membrane-permeable, small molecules that bind the actin binding site on the B subunit, using computational chemistry techniques. To perform comparative structural modeling to generate an atomic-level model of the B2 subunit, 3D-PSSM and Swiss Model were used. The approach utilized: (1) generation of a multiple alignment with the sequence to be modeled (Guex, N., A. Diemand, et al., Protein modelling for all. Trends Biochem. Sci. 1999, 24:364-367), (2) generation of a framework for the new sequence based on superposition of the related three-dimensional structure, (3) rebuilding loops and side chains (Peitsch, M. C., et al. 2000. Automated protein modelling—the proteome in 3D. Pharmacogenomics. 2000, 1:257-266), (4) structural refinement by energy minimization and molecular dynamics (Schwede, T., et al. 2000. Protein structure computing in the genomic era. Res. Microbiol. 151:107-112), and (5) verification of the model structure geometry (Schwede, T., et al. 2003. SWISS-MODEL: An automated protein homology-modeling server. Nucleic Acids Res. 31:3381-3385).

Information gleaned by analysis of crystal structures of the profilin 1-actin complex was also used to identify the specific binding pockets of subunit B2 that were most likely required for the interaction with actin. It was determined that the actin binding region of subunit B2 shares sequence homology with the actin binding site of profilin 1, and the actin binding site of B2 and profilin 1 compete when binding actin (Chen, S. H., et al. J. Biol. Chem. 2004, 279:7988-7998), and it was found that the actin binding surfaces were likely similar.

Molecular docking simulations where each one of approximately 300,000 small molecules (mw<500 with properties consistent with membrane-permeability) were positioned in the selected structural pockets and scored based on predicted polar and non-polar interactions were performed for the B2 subunit model. The molecular surface of the structure was explored using sets of spheres to describe potential binding pockets. The SPHGEN program generates an unbiased grid of points that reflect the actual shape of the selected site. The CLUSTER program groups the selected spheres to define the points that are used by DOCK to match (superimpose) potential ligand atoms with spheres (Ewing, T. J., et al. J. Comput. Aided Mol. Des 2001, 15:411-428). Each compound in the NCI/DTP database was positioned in the selected site in approximately 100 different orientations. Intermolecular AMBER energy scoring (vdw+columbic), contact scoring and bump filtering were implemented in DOCKv6.1.0. (Perola, E., et al. Proteins, 2004, 56:235-249).

From an initial virtual screen, 100 small molecules (ranked 1-100 regarding best fit score) were identified. Forty small molecules were obtained from the repository at the Drug Synthesis and Chemistry Branch of the National Osteoclast-related disease Institute. Certain compounds of the invention are synthesized within the means of chemists of ordinary skill in the art (see, e.g., Herczegh et al., Osteoadsorptive Bisphosphonate Derivatives of Fluoroquinolone Antibacterials, J. Med. Chem. 2002, 45, 2338-2341).

### Example 3

Twenty-one small molecules are tested for their ability to block interaction between recombinantly-expressed B2 subunit and pure rabbit muscle F-actin. These compounds are 1) {3,5,7-Triaza-1-azoniatricyclo[3.3.1.13,7]decane}{1-[(2,}4,5-trichlorophenyl)methyl]-, chloride (“Binhib 1”); 2) 3,5,7-triaza-1-azoniatricyclo[3.3.1.13,7]decane, 1-[2-(4-chlorophenyl)-2-oxoethyl]-iodide (“Binhib 2”); 3) 1′,2′,10,13,17-pentamethylhexadecahydrospiro[cyclopenta[a]phenanthrene-3,3′-diaziridin]-17-ol (“Binhib 3”); 4) ethyl 2-((2-aminoethyl)amino)-3-(1H-indol-3-yl)propanoate (“Binhib 5”); 5) 1-ethyl-6-fluoro-4-oxo-7-(piperazin-1-yl)-1,4-dihydro-1,8-naphthyridine-3-carboxylic acid (“Binhib 7” or “enoxacin”); 6) 3-(5,7-Triaza-1-azoniatricyclo[3.3.1.13,7]decane) (1-[(4-bromophenyl)methyl])bromide (“Binhib 9”); 7) 1-(4-iodophenyl)-2-(15,3,5,7-tetraazatricyclo[3.3.1.1˜3,7˜]dec-1-yl)ethanone oxime (“Binhib 10”); 8) 2-((4-methyl-1-piperazinyl)methyl)-15-indeno[1,2,3-de]quinazolin-1-ol (“Binhib 15”); 9) 4-(3-methoxybenzyl)-1-piperazinamine (“Binhib 17”); 10) 1-(2,3-dibromo-2-propenyl)-15,3,5,7-tetraazatricyclo[3.3.1.1˜3,7˜]decane (“Binhib 18”); 11) 1-(4-isobutylbenzyl)-15,3,5,7-tetraazatricyclo[3.3.1.1˜3,7˜]decane (“Binhib 20”); 12) ethyl 3-((2-chloro-5-(hydroxy(oxido)amino)-6-methylhexahydro-4-pyrimidinyl)amino)propanoate (“Binhib 23”); 13) 1-(4-bromophenyl)-2-(15,3,5,7-tetraazatricyclo[3.3.1.1˜3,7˜]dec-1-yl)ethanone (“Binhib 24”); 14) 1-(2-hydroxy-3a,12a-dimethyl-7-(methylamino)-6-methylenehexadecahydro-cyclopenta[a]cyclopropa[e]phenanthren-1-yl)ethanone (“Binhib 26”); 15) 1-((2-methyl-3-quinolinyl)methyl)-15,3,5,7-tetraazatricyclo[3.3.1.1˜3,7˜]decane (“Binhib 30”); 16) 8,8′-disulfanediylbis(1,3-dimethyl-1H-purine-2,6(3H,9H)-dione) (“Binhib 31”); 17) 2,4-diamino-5-(4-((2-aminoethyl)amino)-3-(hydroxy(oxido)amino)phenyl)-6-ethylpyrimidine (“Binhib 33”); 18) methyl 1-formyl-17-methoxyaspidofractinine-3-carboxylate (“Binhib 35”); 19) 1-(2-naphthyl)-2-(15,3,5,7-tetraazatricyclo[3.3.1.1˜3,7˜]dec-1-yl)ethanone (“Binhib 36”); 20) 2-(1-piperazinyl)ethylcarbamodithioic acid (“Binhib 38”); and 21) 1-(4-methoxyphenyl)-2-(15,3,5,7-tetraazatricyclo[3.3.1.1˜3,7˜]dec-1-yl)ethanone (“Binhib 41”).

Recombinant B subunit (1 μM) plus/minus F-actin (2 μM)) plus/minus 100 uM inhibitor compounds were incubated for 10 minutes in 20 mM Tris/HCl (pH 7.5), 100 mM NaCl, 1 mM MgCl₂, 0.2 mM CaCl₂, 0.5 mM ATP, 0.2 mM DTT, then subjected to ultracentrifugation at 180,000×g for 45 min. Supernatants and pellets were collected in SDS-PAGE sample buffer, separated by SDS-PAGE, stained with Coomassie Blue, and densitometry was performed using a Flurchem 8000 (Alpha Innotech, San Leandro Calif.).

The results are summarized in Table 1 (see below). The percent inhibition was determined by densitometry of SDS-PAGE gels. The raw densitometric number obtained of the recombinant B subunit band that was pelleted with F-actin, but with no compound present, represented 0% inhibition. The amount of the raw densitometric number of the amount of recombinant B subunit that pelleted with no actin present, but with compound, represented 100% inhibition. The amount of recombinant B subunit that pelleted with actin and the compound being tested was placed as a percentage assuming a linear response. Positive results were confirmed by additional pelleting assays (see Holliday, L. S., et al. J. Biol. Chem. 2000, 275:32331-32337).

TABLE 1

| Compound | % inhibition |
|---|---|
| Binhib 23 | 2 |
| Binhib 2 | 65 |
| Binhib 33 | 1 |
| Binhib 30 | 4 |
| Binhib 17 | 0 |
| Binhib 7 | 92 |
| (also as “enoxacin”) | |
| Binhib 18 | 2 |
| Binhib 1 | 5 |
| Binhib 15 | 0 |
| Binhib 24 | 13 |
| Binhib 9 | 21 |
| Binhib 5 | 0 |
| Binhib 38 | 3 |
| Binhib 41 | 4 |
| Binhib 10 | 3 |
| Binhib 35 | 4 |
| Binhib 3 | 7 |
| Binhib 26 | 0 |
| Binhib 31 | 2 |
| Binhib 36 | 0 |
| Binhib 20 | 5 |

### Example 4

Based on the assays above-described, enoxacin (listed as “Binhib 7” in Table 1) and Binhib 2 were identified to block greater than 90% of binding at the B subunit-F-actin at a concentration of 100 μM.

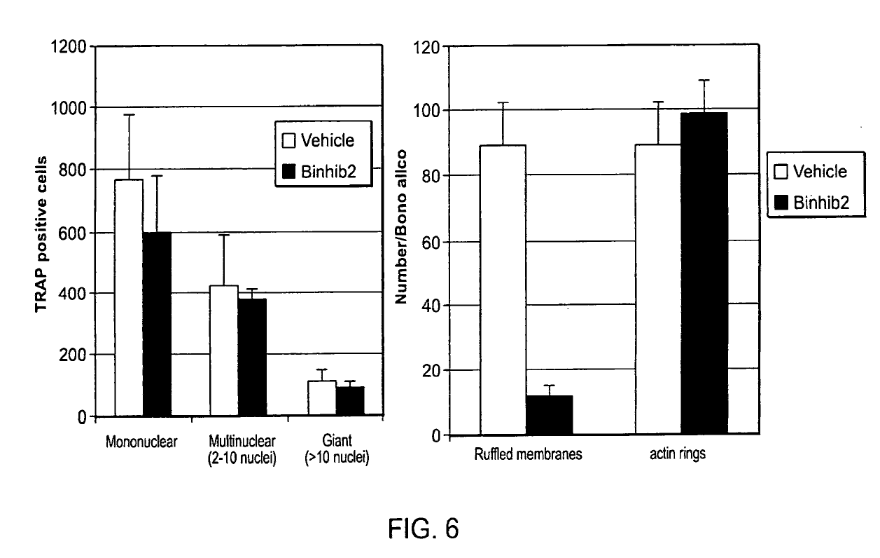

Enoxacin and Binhib 2 were then tested for their effects on osteoclastogenesis stimulated by calcitriol in mouse marrow cultures. The mixed cultures represent a model for the complex interactions that regulate bone resorption in vivo. The results for enoxacin are listed in FIG. 5, and test results for Binhib 2 are demonstrated in FIG. 6.

FIG. 5 shows that enoxacin inhibited osteoclast formation with an IC₅₀ of 1 μM, and that 10 μM enoxacin completely inhibited V-ATPase trafficking to ruffled membranes in mature osteoclasts. In addition, at 1-10 μM enoxacin, osteoclast formation was inhibited while osteoblast formation, judged by alkaline phosphatase expressing cells, was not affected. And, at level of 100 μM, enoxacin blocked 90% of the B subunit from binding F-actin. It also shows that enoxacin concentrations as low as 1 μM significantly reduced the formation of osteoclasts. In addition, it was found that even single doses of enoxacin at the onset of the marrow culture period resulted in dramatic reductions in osteoclast formation at the end of 7 Days (Data not shown). These results suggest that enoxacin affects the capacity of osteoclast precursors to be related down the differentiation pathway toward osteoclasts in these cultures.

FIG. 6 shows that Binhib 2 inhibits B subunit actin interactions in vitro, blocks ruffled membrane formation (right panel), without affecting osteoclast formation (left panel).

### Example 5

The present inventors also tested the effects of enoxacin on osteoclasts that were already mature and loaded onto bone slices. In this experiment, mature osteoclasts on bone slices were treated with enoxacin or vehicle control for 3 days, then fixed and labeled with an anti-E subunit antibody. Control osteoclasts produced many ruffled membranes. But in enoxacin-treated cultures, E subunit labeling was detected in a perinuclear distribution.

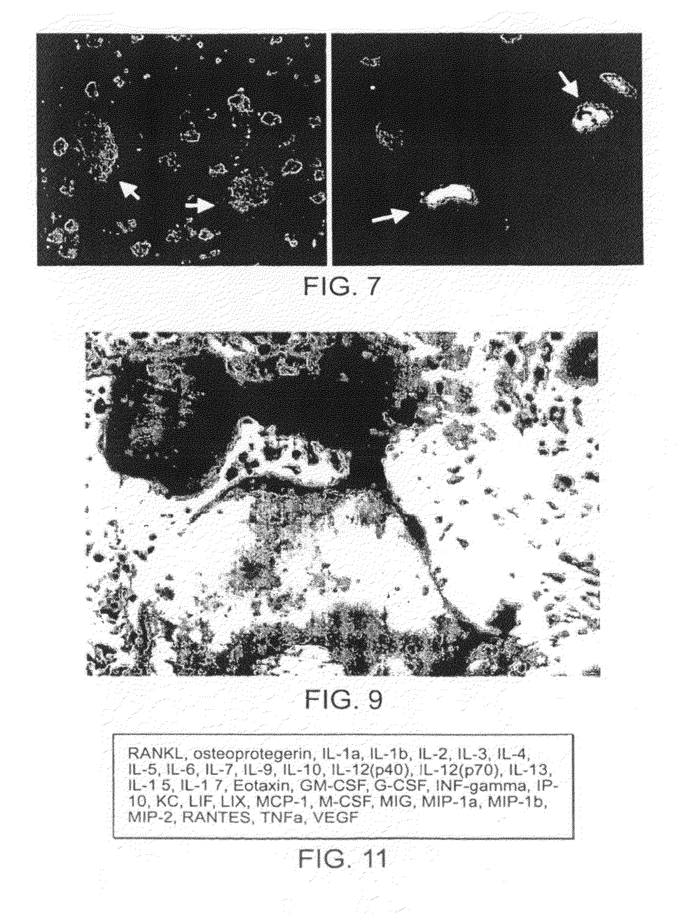

The test results are depicted in FIG. 7, which shows that enoxacin (10 μM) blocks ruffled membrane formation. Arrows point to representative osteoclasts.

### Example 6

MiRNAs were isolated from RAW 264.7 cells before or after stimulation to form osteoclast-like cells by treatment with RANKL. The isolated miRNAs were submitted to LC Sciences (Houston, Tex.) where microarrays for miRNAs were performed. Microarrays have been widely used to monitor changes in gene expression.

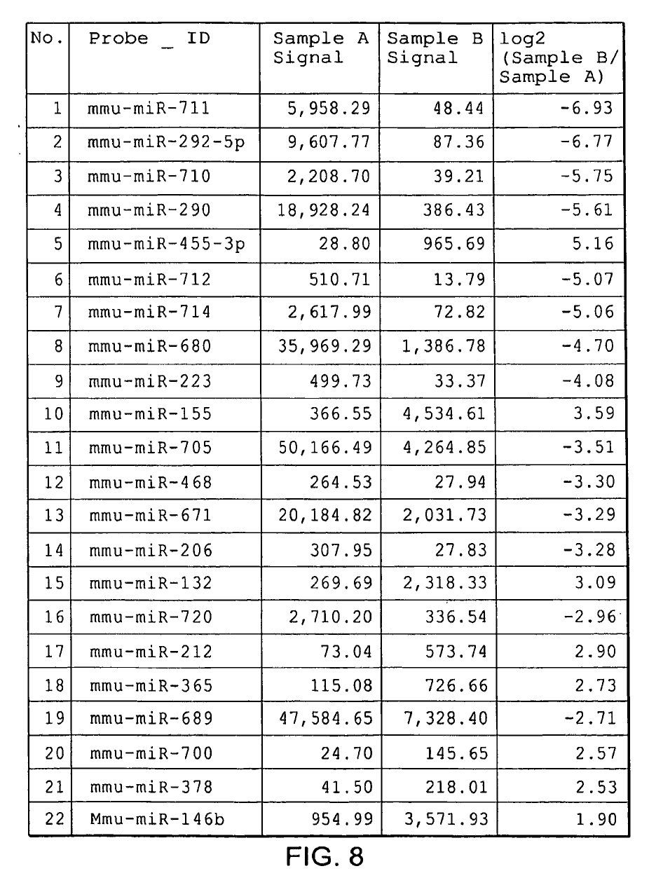

This study revealed alterations in a number of miRNAs occurred in the RAW cells in response to RANKL. These data are summarized in FIG. 8.

### Example 7

Primary mouse marrow osteoclasts are grown by a slight modification of the method used by Feng and colleagues (Feng, Y., et al. J. Biol. Chem., 2007, 282:39-48). Mouse marrow from 5 mice is collected and incubated for one day in αMEM plus 10% fetal bovine serum (αMEM D10). The next day, non-adherent cells are collected, spun and replated in a fresh tissue culture plate supplemented with 1 ml of conditioned media from the ongoing mouse marrow culture from where the non-adherent cells were derived. The conditioned media provides a convenient, low cost source of macrophage-colony stimulating factor (CSF-1). The cells grow for 5-6 days with supplementation of 1 ml of whole mouse marrow conditioned media. The osteoclast precursors then are scraped and loaded onto acid washed coverslips, bone slices or into fresh tissue culture plates at a density of 1.5×10⁵ cells/cm² and these cultures are supplemented with conditioned media and recombinant RANKL (5 ng/ml). During the next 4-6 days osteoclasts differentiate (FIG. 9).

Osteoclast formation: Hematopoietic precursors are loaded into 24 well plates, treated with RANKL and concentrations of enoxacin (0.001 μM-100 μM) or vehicle control on Day 1 and this will be refreshed on Day 3 of incubation. On Day 6, the cells are fixed with 2% formaldehyde and stained for TRAP activity (Holliday, L. S., et al. Am. J. Physiol, 1997, 272:F283-F291), or with phalloidin to detect actin belts (Holliday, L. S., et al. Interstitial Collagenase Activity Stimulates the Formation of Actin Rings and Ruffled Membranes in Mouse Marrow Osteoclasts. Calcif. Tissue Int. 2003). The numbers of TRAP+ multinucleate cells and multinuclear cells exhibiting actin belts are enumerated by counters who are blinded to the treatment conditions (typically undergraduate volunteers). In each experiment, 4-6 wells per treatment group are used, and each experiment is repeated independently 3 times or more.

Osteoclast protein expression: The effects of enoxacin are examined on the expression of several proteins that are normally upregulated as osteoclasts differentiate. The primary osteoclast precursors are plated in 6 well plates and treated with a range of concentrations of enoxacin or vehicle control. After 6 Days the cells are washed several times in PBS, then collected in 250 μl Laemmli sample buffer and subjected to ultracentrifugation (100 K×g/20 min), which pellets nucleic acids that interfere with SDS-PAGE. The extracted proteins are separated by SDS-PAGE (proteins will be loaded at several loading concentrations), blotted to nitrocellulose, and probed with antibodies to subunit a3, E and B2 of V-ATPase, cortactin, Arp3 (Santa Cruz) and cathepsin K (Santa Cruz). Blots are also stained with an anti-actin antibody (Sigma) as a loading control. By comparing different loading concentrations of each sample, the relative expression of the target proteins is determined, despite the fact that the Western blot detection system does not respond in a linear manner over a wide range of protein concentrations.

Osteoclast bone resorption: Primary osteoclasts are grown in 100 cm plates, then, after 5 days, scraped and loaded atop bone slices. In some experiments, cells are treated with enoxacin (100-0.001 μM) or vehicle for the entire differentiation and resorption period. In other studies, the effects of enoxacin on the preformed osteoclasts are tested, adding the compound at various concentrations only after the osteoclasts are loaded onto bone slices. In some cases the osteoclasts and assay are fixed for actin ring formation (staining with fluorescently-tagged phalloidin), ruffled membrane formation (making use of our anti-E subunit antibody) and TRAP activity (after actin rings and ruffled membranes have been counted and photographically documented).

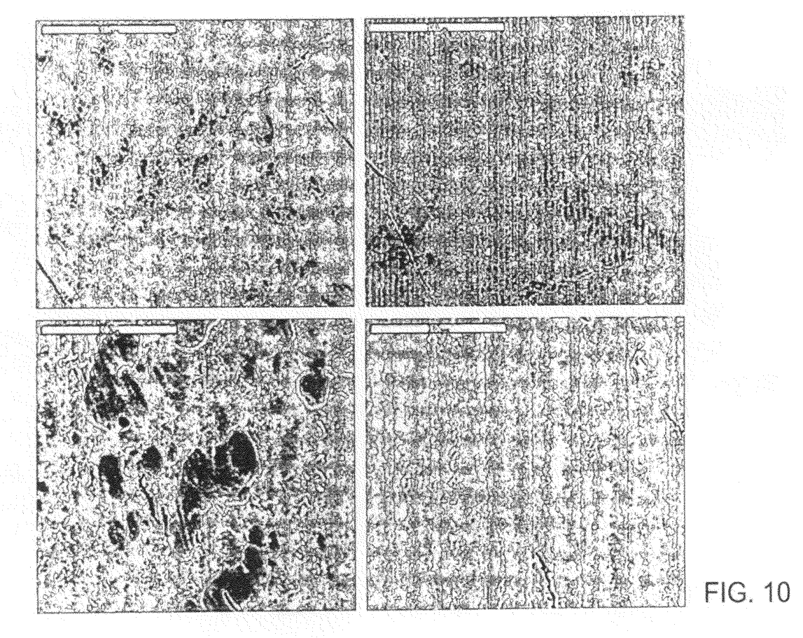

In some experiments, osteoclast resorption is determined based on pit formation (Holliday, L. S., et al. J. Biol. Chem. 1995, 270:18983-18989). Cells are removed from bone slices after the incubation period using 1% SDS in water. Slices will dehydrated through an ethanol series, mounted on stubs and examined using SEM (FIG. 10). Electronic images are analyzed using Image J (Downloaded from NIH website).

Data collected include the total surface, the area resorbed, the pit size and the number of pits. As described above, each experiment has a N of 4-6 and is repeated independently at least 3 times.

Osteoblast Expression of RAN KL, Osteoprotegerin, Alkaline Phosphatase and Other Regulatory Molecules:

Osteoclast and osteoblast-containing mouse bone marrow cultures are obtained as above described. 4-6-week-old Swiss-Webster mice (Harlan, Indianapolis, Ind.) are sacrificed by cervical dislocation. Femurs and tibias are dissected free of adherent tissue; the marrow is expelled by cutting both bone ends and flushing the marrow cavity with MEM D10 using a 25-gauge needle. The marrow cells are washed twice and plated in 6 well plates at a concentration of 1×10⁶ nucleated cells/cm² in MEM D1 0 containing 10 nM calcitriol. Cultures are fed on the third and fifth day of culture by replacing half of the medium with fresh medium containing calcitriol. Cultures are treated with enoxacin at 10 μM or vehicle control on Day 1, and when the media is refreshed.

Osteoblast-enriched cultures are obtained by culture of mouse calvaria (Cowan, C. M., et al. J. Biol. Chem., 2003, 278:32005-32013). Calvaria is dissected from 7-day-old pups. Calvarial cells are isolated by four sequential 15-minute digestions in an enzyme mixture containing 0.05% trypsin (Gibco BRL, Rockville, Md., USA) and 0.1% collagenase P (Boehringer Mannheim, Indianapolis, Ind., USA) at 37 collected, resuspended in media, and plated at 5×10³ cells/cm² Dulbecco's modified Eagle's medium (DMEM; Gibco BRL) containing 10% fetal calf serum (FCS; Gibco BRL). After the cells reach a confluent monolayer at day 7, α-MEM (Gibco BRL) containing 10% FCS, 50 μg/mL ascorbic acid (Gibco BRL), and 5 mmol β-glycerophosphate (Gibco BRL) is used to maintain the cells for the duration of the experiment. Cells are harvested for analysis at different time points: Day 6 or 7, 12, 18, or 25. Cultures are treated with enoxacin (0.001 μM-100 μM) or vehicle control on Day 1, and the media is refreshed every third day of the culture period.

In the case of both calcitriol stimulated mouse marrow and calvarial osteoblast cultures, at the end of the incubation period cell extracts are prepared exactly as described in the protocols for the mouse cytokine and chemokine multiplex kits, mouse RANKL and mouse OPG 1 plex kits (Millipore) (FIG. 11).

Osteoblast growth and survival: Primary calvarial osteoblasts are plated in a 96-well assay plate in media at a concentration of 30000 cells per well. Enoxacin (100-0.001 μM) or vehicle are added to each well in a total volume of 200 μl of media. The cells are incubated at 37° C. for 40 h. To each well, 40 μl of CellTiter 96 Aqueous One Solution Reagent are added and the cells are incubated for 4 h at the same conditions mentioned previously. The amount of soluble formazan produced by cellular reduction of the MTS is measured and recorded the absorbance at 490 nm using a 96-well plate reader. In this assay, the absorbance is directly proportional to cell proliferation. All experiments are performed in triplicate.

Osteoblast mineralization assays: To obtain bone marrow stromal cell (MSC) cultures, marrow is flushed from tibiae and femora of 6-8 week-old mice with α-MEM (Invitrogen, Carlsbad, Calif.). 1×10⁶ cells/well is plated in 6 well dishes and cultured for up to 21 days. For calvarial osteoblast (COB) cultures, calvariae are dissected from 3-5 neonatal mice and digested with 0.5 mg/ml of collagenase P (Roche Diagnostics, Indianapolis, Ind.) in a solution of 1 ml trypsin/EDTA and 4 ml PBS at 37° C. Four digests are performed for 10 min and a final digest for 90 min. Digests 2-5 are pooled and plated at 4×10⁴ and cultured for up to 21 days.

Culture medium is α-MEM with 10% fetal calf serum, 100 U/ml penicillin, 50 μg/ml streptomycin and 50 μg/ml phosphoascorbate (Sigma). Cells are cultured in a humidified atmosphere of 5% CO2 at 37° C. with media changes every 3-4 days. 10 mM of β-glycerophosphate is added to the medium on day 7 for the duration of the experiment. Vehicle, or enoxacin (100-0.001 μM) or vehicle control is added to control cultures. To assess mineralization, cells are washed with PBS, fixed in 100% V/V methanol on ice for 30 min and stained with 40 mM alizarin red-S (Sigma) pH 4.2 for 10 min at room temperature. Dishes are washed with water, air dried and scanned into the computer.

### Example 8

Mouse marrow osteoclasts are differentiated on 6-well plates by stimulation with recombinant RANKL as described above. Cells are treated with enoxacin (0.001-100 μM) or vehicle control.

Detergent-insoluble cytoskeleton: At the end of the incubation period, the cells are solubilized in 1% Triton X-100 and subjected to ultracentrifugation. Supernatants and pellets are collected, separated by SDS-PAGE, blotted and probed with antibodies against V-ATPase subunits a3 and E. The relative amounts of these V-ATPase subunits associated with detergent-insoluble “cytoskeletal” fraction in the presence of various concentrations of enoxacin or vehicle are examined.

In follow up experiments, control detergent-insoluble cytoskeletal fractions are treated with various concentrations of enoxacin or vehicle for 1 h prior to pelleting to determine if this releases the V-ATPase from the detergent-insoluble cytoskeleton in cell extracts. Each experiment is performed at least 3 times.

Cytological techniques are used to determine whether enoxacin disrupts the association of V-ATPase with the detergent-insoluble cytoskeleton. Osteoclasts are allowed to mature on coverslips, then treated with extraction buffer (20 mM Tris (pH 7.5), 100 mM NaCl, 5 mM MgCl₂, 0.2 mM CaCl₂ Triton X-100 and 0.01 mg/ml rhodamine-tagged phalloidin). After 10 mM on ice, the extracted remnants are fixed in ice-cold 2% formaldehyde for 20 min and then incubated in blocking solution followed by anti-E subunit antibody and fluorescein isothiocyanate-conjugated anti-mouse antibody. Specimens are examined by a spinning disc scanning laser confocal microscopy. In some experiments, it is also tested whether the addition of enoxacin to the extraction buffer removes the V-ATPase from the detergent-insoluble cytoskeleton of vehicle treated osteoclasts.

Immunoprecipitations: To further test whether enoxacin disrupts the capacity of V-ATPase to interact with microfilaments, immunoprecipitations are performed from soluble extracts of osteoclasts that had been treated with enoxacin or vehicle control using anti-E subunit and anti-a3 subunit antibodies developed by the present inventors (Zuo, J., et al. J. Bone Miner. Res. 2006, 21:714-721; Lee, B. S., et al. J. Bone Miner. Res., 1999, 14:2127-2136). It is expected that treatments of control extracts with enoxacin after cells are homogenized, but prior to immunoprecipitation, reduces the amount of F-actin attached to the immunoprecipitated V-ATPase.

Ruffled membrane formation: In the studies, cultures that were treated with enoxacin at the same time were examined that they were loaded atop bone slices. In order to more precisely isolate effects on mature osteoclasts, the cultures are differentiated and loaded onto bone slices and allowed to activate to resorb. Enoxacin is then added to the cultures for times ranging from 10 min to 4 h. The osteoclasts are fixed and examined for actin ring and ruffled membrane formation. It is expected that osteoclasts go through cycles of resorption and resolution that are in the range of hours long.

Preliminary data indicated that ruffled membrane formation was disrupted by the treatment of differentiated osteoclasts with enoxacin.

### Example 9

This example tested whether enoxacin alters miRNA activity, and whether this is a mechanism by which osteoclast formation and bone resorption are inhibited.

To determine whether stimulation of miRNAs effect osteoclasts, the effects of stimulators of miRNAs with related agents are compared to those do not affect miRNAs. Assay GW bodies, and presumptive markers for miRNAs are used. Further, expression levels of a protein are examined that are predicted to decrease in expression due to stimulation of miRNA activity.

Structure function studies: Use a series of small molecules that are reported to 1) stimulate miRNA activity, 2) to be structurally-similar but not stimulate miRNA activity, or which block V-ATPase-actin interactions but are not reported to stimulate miRNAs, and 3) which have no known effects on osteoclasts. These molecules are tested in assays for osteoclast formation and ruffled membrane formation and bone resorption as described above.

GW bodies: GW bodies have been reported to be correlated with miRNA activity (Pauley, K. M., et al. EMBO Rep. 2006, 7:904-910; Lian, S., et al. Cell Cycle 2006, 5:242-245). Enoxacin, an enhancer of miRNA activity, is predicted to increase the number of GW bodies present and/or increase the size of GW bodies.

To test whether enoxacin affects GW body formation, calcitriol-stimulated mouse marrow cultures, RANKL-stimulated primary hematopoietic cells and primary calvarial osteoblasts are treated with enoxacin. A dose response curve using doses above and below the IC50 dose is performed for inhibition of osteoclast formation and bone resorption. Osteoclast formation, staining and enumerating TRAP+ cells, and ruffled membrane formation are examined, by treating preformed osteoclasts on bone slices. Cells are fixed at various time points after treatment with enoxacin, and stain with polyclonal antibody against GW bodies and monoclonal anti-DCP1. The number of GW bodies in at least 100 randomly sampled cells is counted. The number of GW bodies is divided by the number of nuclei/cell, as determined by counting DAPI stained nuclei. This experiment is repeated in triplicate. Counts are performed by pre-calibrated assistants who are blinded to the nature of the experiment. Images are taken using a Zeiss Axiovert 200 M microscope and a Zeiss AxioCam MRm camera using the 40×0.75 NA objective.

Changes in protein levels: Numerous miRNAs are present in osteoclast precursors and osteoclasts, fewer, but still a significant number, are either up- or down-regulated during the course of osteoclastogenesis, and each miRNA may have many mRNA targets. This is to focus on a validated miRNA-based regulatory pathway that is predicted to be relevant for osteoclasts.

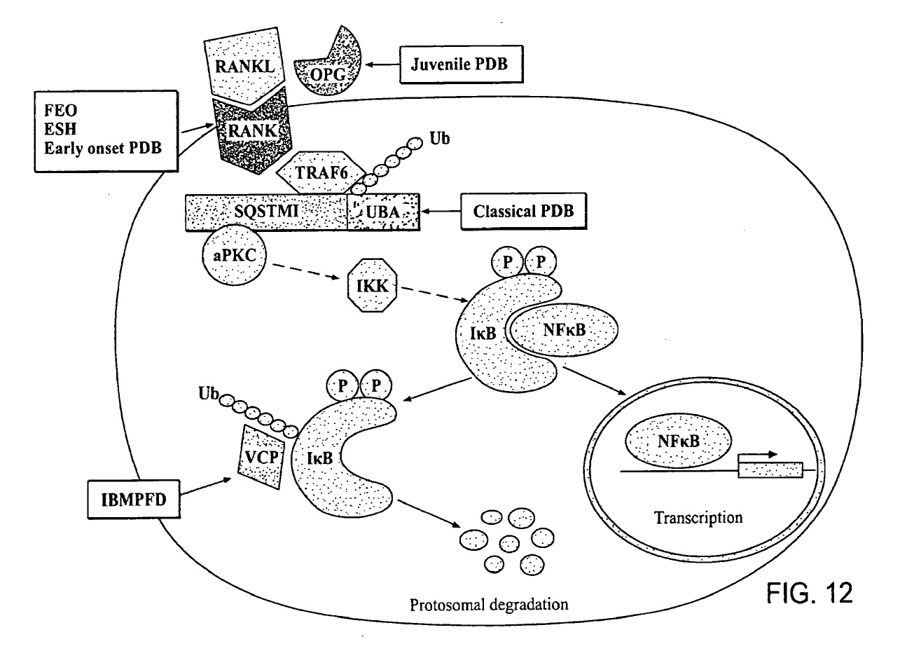

A number of previous studies have found that miR-155 is upregulated in response to stimulation of NF kappa B by toll-like receptors. Upregulation of miR-155 leads to attenuation of NF kappa B-signaling, and a miR-155 knockout mouse displays abnormalities in the immune system (Lu, F., et al. 2008. EBV Induced miR155Attenuates NF-{kappa} B Signaling And Stabilizes Latent Virus Persistence. J. Virol.; Thai, T. H., et al. Science 2007, 316:604-608). The mechanism involves repression of IKKepsilon, which is a critical element of the pathways by which both Toll-like receptors and RANKL utilize to stimulate NF Kappa B (Lu, F., et al. 2008. EBV Induced miR155 Attenuates NF-{kappa}B Signaling And Stabilizes Latent Virus Persistence. J. Virol.). Enoxacin is predicted to increase the activity of miR-155, further attenuate NF kappa B signaling, and reduce osteoclast formation, which is dependent on this pathway (FIG. 12).

Levels of IKKepsilon using anti-IKKepsilon (IMGENEX) for quantitative Western blotting are monitored after a treatment with enoxacin. Anti-actin and anti-GAPDH are used as internal controls.

### Example 10

Enoxacin is tested in ovariectomized (OVX) rats that lose bone in a manner similar to that of postmenopausal women. This model has been widely and successfully utilized as an early pre-clinical model for osteoporotic disease (Pun, S., et al. Bone, 2000, 27:197-202; Wronski, T. J., et al. Endocrinology, 1993, 132:823-831; Wronski, T. J., et al. Endocrinology, 1988, 123:681-686; Iwaniec, U. T., et al. Endocrinology, 2002, 143:2515-2526). In this study, the effects of systemic administration of enoxacin are examined.

In this study, ligands identified that block V-ATPase/F-actin interactions and bone resorption in vitro are advanced to in vivo testing in OVX rats. Female Sprague Dawley rats are subjected to sham surgery or ovariectomized at 3 months of age (see the preceding references). On the day after surgery, groups of sham operated control and OVX rats are injected subcutaneously (SC) with vehicle or treated with vehicle via oral gavage. A second group of OVX rats are injected SC 3 days/week with 17β-estradiol (Sigma, St. Louis, Mo.) at a dose of 10 μg/kg to inhibit bone resorption and prevent cancellous bone loss. The animals are treated orally with daily doses of enoxacin including 10, 100 and 400 mg/kg body weight. Each group consist of 10 rats. All treatments are performed for 6 weeks. Due to the potential for enoxacin to affect intestinal flora, rats are weighed weekly and closely monitored for weight loss and diarrhea. All animals are injected SC with fluorochrome markers (calcein and demeclocycline) at a dose of 15 mg/kg on the 3^{rd} and 10^{th} days before necropsy to label bone forming surfaces.

The animals are sacrificed by exsanguination from the abdominal aorta under ketamine/xylazine anesthesia. The proximal tibiae are collected from each rat for histology. Bone morphometric analyses consist of measurements of bone mass (cancellous bone volume), unmineralized bone mass (osteoid volume) bone formation (osteoblast surface, osteoid surface, mineralizing surface, mineral apposition rate, and bone formation rate), and bone resorption (osteoclast surface). Sections with an anti-E subunit of V-ATPase antibody are stained in order to test on ruffled membrane formation.

In addition to reducing the number of osteoclasts, it is expected that osteoclasts that are present is less active due to inability to properly target V-ATPases to ruffled membranes. Enoxacin treatment triggers the presence of osteoclasts along the bone surface, which does not have ruffled membranes.

### Example 11

Mouse marrow cultures were treated with bis-enoxacin at the concentrations of 5 mM, 10 mM, 25 mM, 50 mM, and 100 mM respectively, for 6 Days in wells of a 24-well plate. The cells were fixed and stained for tartrate-resistant acid phosphatase (TRAP) activity which is a marker for osteoclasts. The number of TRAP positive mononuclear, multinuclear (2-10 nuclei) and giant (>10 nuclei) were counted.

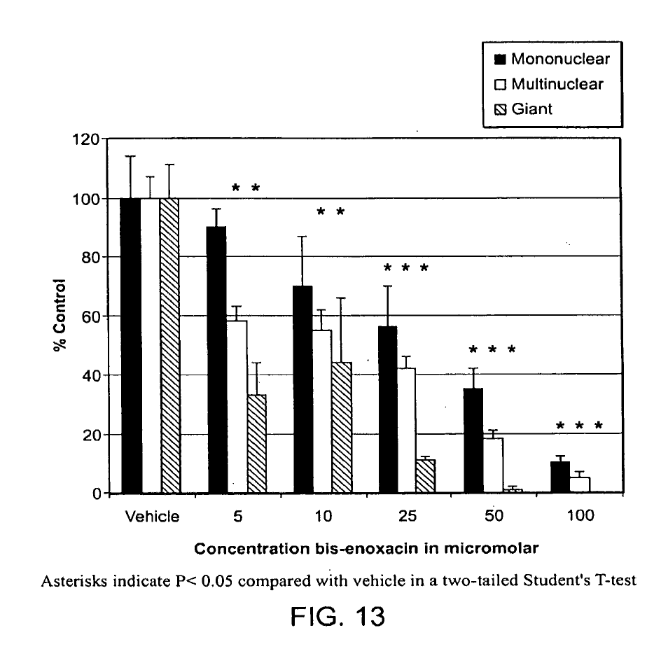

FIG. 13 depicts the effects of bis-enoxacin on mouse marrow cultures. The results demonstrate that bis-enoxacin had identical effects on osteoclast formation as enoxacin.

### Example 12

Bone slices were incubated with bis-enoxacin at the concentrations of 10 mM and 1 mM respectively. After a 1 Day incubation, the slices were washed 4 times over a period of 24H. Mouse marrow osteoclasts were then plated on the slices, and after 3 Days of culture the cells were fixed and the number of actin rings (a marker for active osteoclasts) was counted.

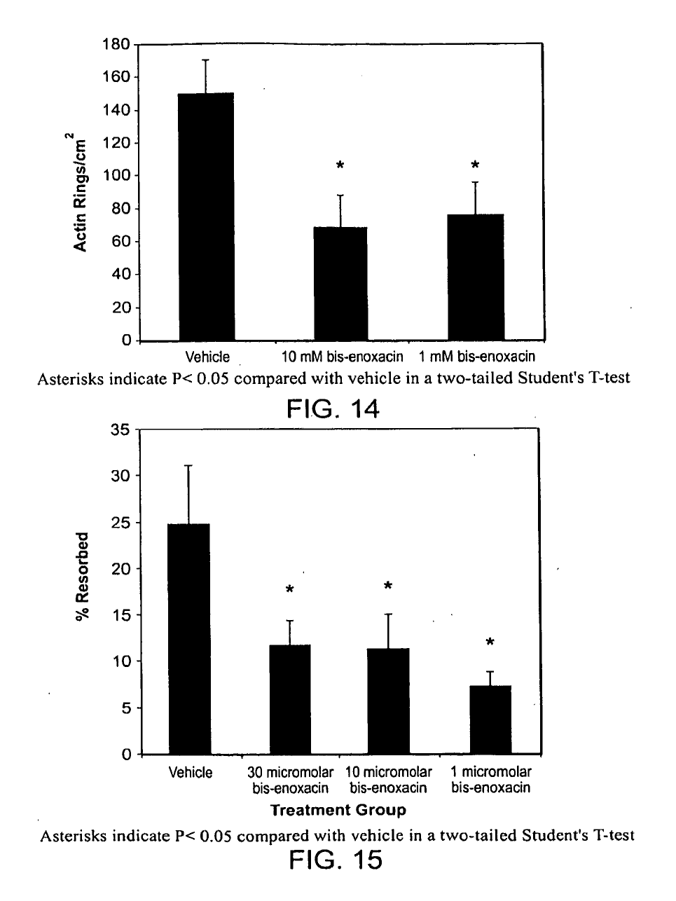

FIG. 14 depicts the effects on actin ring numbers of pre-treating bone slices with bis-enoxacin at concentrations of 10 mM and 1 mM. The pre-treatment with bis-enoxacin reduced actin ring numbers by approximately 60%.

### Example 13

Mouse marrow osteoclasts were grown to maturity for 5 Days then scraped and plated on bone slices treated with bis-enoxacin at the concentrations of 30 mM, 10 mM and 1 mM respectively. After 5 Days, the cells were removed and pitting was quantified by scanning electron microscopy.

FIG. 15 demonstrates that each of the concentrations (30 mM, 10 mM, and 1 mM) of bis-enoxacin reduced bone resorption compared to control, and that there was no statistical significance between the different concentrations of bis-enoxacin. It is believed that essentially all of the bis-enoxacin was bound to the bone, and enough was present associated with the bone, even at the 1 μM starting concentration, to block bone resorption when it was released from bone as osteoclasts began resorbing.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

All references and publications cited herein are hereby incorporated by reference in their entirety.

1. A method for treating or preventing an osteoclast-related disease or disorder in a subject as identified in need of a treatment of a bone disease, said method comprising administering to said subject an effective amount of a compound identified as capable of reducing bone resorption and stimulating new bone formation.

2. The method of claim 1, wherein said compound is capable of inhibiting or interfering with interaction between V-ATPase and F-actin in the membranes of osteoclasts.

3. The method of claim 2, wherein said compound is selected from the group of fluoroquinolone derivatives and bis-phosphonate derivatives, or a pharmaceutically acceptable salt, solvate, clathrate, hydrate, polymorph, or prodrug thereof.

4. The method of claim 3, wherein said compound is selected from the group of 1-ethyl-6-fluoro-4-oxo-7-(piperazin-1-yl)-1,4-dihydro-1,8-naphthyridine-3-carboxylic acid (“enoxacin”); 1-cyclopropyl-6-fluoro-4-oxo-7-piperazin-1-yl-quinoline-3-carboxylic acid (“ciprofloxacin”); 1-ethyl-6-fluoro-4-oxo-7-(piperazin-1-yl)-1,4-dihydroquinoline-3-carboxylic acid (“norfloxacin”); 1-ethyl-6-fluoro-7-(4-methylpiperazin-1-yl)-4-oxo-1,4-dihydroquinoline-3-carboxylic acid (“pefloxacin”); 3,5,7-triaza-1-azoniatricyclo[3.3.1.13,7]decane, 1-[2-(4-chlorophenyl)-2-oxoethyl]-, iodide (“Binhib 2”); and 7-(4-(2,2-diphosphonoethyl)piperazin-1-yl)-1-ethyl-6-fluoro-4-oxo-1,4-dihydro-1,8-naphthyridine-3-carboxylic acid (“bis-enoxacin”), or a pharmaceutically acceptable salt, solvate, clathrate, hydrate, polymorph, or prodrug thereof.

5. The method of claim 4, wherein said compound is 1-ethyl-6-fluoro-4-oxo-7-(piperazin-1-yl)-1,4-dihydro-1,8-naphthyridine-3-carboxylic acid (“enoxacin”), or 7-(4-(2,2-diphosphonoethyl)piperazin-1-yl)-1-ethyl-6-fluoro-4-oxo-1,4-dihydro-1,8-naphthyridine-3-carboxylic acid (“bis-enoxacin”), or a pharmaceutically acceptable salt, solvate, clathrate, hydrate, polymorph, or prodrug thereof.

6. The method of claim 5, wherein said compound is 7-(4-(2,2-diphosphonoethyl)piperazin-1-yl)-1-ethyl-6-fluoro-4-oxo-1,4-dihydro-1,8-naphthyridine-3-carboxylic acid (“bis-enoxacin”), or a pharmaceutically acceptable salt, solvate, clathrate, hydrate, polymorph, or prodrug thereof.

7. The method of claim 2, wherein said compound is capable of stimulating microRNA activity.

8. The method of claim 7, wherein said compound is 1-ethyl-6-fluoro-4-oxo-7-(piperazin-1-yl)-1,4-dihydro-1,8-naphthyridine-3-carboxylic acid (“enoxacin”), or 7-(4-(2,2-diphosphonoethyl)piperazin-1-yl)-1-ethyl-6-fluoro-4-oxo-1,4-dihydro-1,8-naphthyridine-3-carboxylic acid (“bis-enoxacin”), or a pharmaceutically acceptable salt, solvate, clathrate, hydrate, polymorph, or prodrug thereof.

9. The method of claim 1, wherein said osteoclast-related disease or disorder is selected from the group of periodontal disease, non-malignant bone disorders, osteoporosis, Paget's disease of bone, osteogenesis imperfecta, fibrous dysplasia, and primary hyperparathyroidism, estrogen deficiency, inflammatory bone loss, bone malignancy, arthritis, osteopetrosis, hypercalcemia of malignancy (HCM), osteolytic bone lesions of multiple myeloma and osteolytic bone metastases of breast cancer, and metastatic cancers, bone cancers, and osteoclast-related dental diseases or disorders.

10. The method of claim 9, wherein said osteroclast-related disease or disorder is selected from the group of osteoporosis, bone cancers, and osteoclast-related dental diseases or disorders.

11. The method of claim 9, wherein said osteroclast-related dental disease or disorder is selected from the group of P. gingivalis infection, periodontal diseases, endodontic diseases/disorders, and orthodontic tooth movement.

12. The method of claim 9, wherein said osteroclast-related disease or disorder is a bone cancer.

13. The method of claim 1, wherein said method further comprises treating said subject with an effective amount of an additional therapeutic agent selected from the group of bisphosphonates, calcitonin, intermittent PTH, and Denosumib, or a mixture thereof.

14. (canceled)

15. A kit used for treating or preventing an osteoclast-related disease or disorder in a subject identified as in need of a treatment of a bone disease, said kit comprising an effective amount of a compound capable of reducing bone resorption and stimulating new bone formation, and instructions for use of said kit.

16. A method for treating or preventing an osteoclast-related disease or disorder in a subject identified as in need of a treatment of a bone disease, said method comprising a) identifying or selecting the subject suffering from or susceptible to an osteoclast-related bone disease or disorder for treatment with a compound, wherein said compound is capable of reducing bone resorption and stimulating new bone formation; b) monitoring the progress of said subject being treated; and c) assessing the efficacy of said treatment in said subject.

17. A packaged composition for treating or preventing an osteoclast-related disease or disorder in a subject identified as in need of a treatment of bone disease, said composition comprising an effective amount of a compound identified as capable of reducing bone resorption and stimulating new bone formation, and a pharmaceutically acceptable carrier or diluent.

18. The packaged composition of claim 17, further comprising instructions to treat an osteoclast-related disease or disorder in said subject.

19. (canceled)

20. The packaged composition of claim 17, wherein said compound is selected from the group of enoxacin, ciprofloxacin, norfloxacin, pefloxacin, Binhib2, and bis-enoxacin, or a pharmaceutically acceptable salt, solvate, clathrate, hydrate, polymorph, or prodrug thereof.
